SEQUENCE LISTING (67 sequences)

SEQ ID NO: 1 - protein, 162 aa, synthetic construct
MKITLRMMVLAVLTAMAMVLAACGGGGSSGGSTGGGSGSGPVTIEIGSKGEELAFDKTELTVSAGQTVTIRFKNNSAVQQHNWILVKGGEAEAANIANAGLSAGPAANYLPADKSNIIAESPLANGNETVEVTFTAPAAGTYLYICTVPGHYPLMQGKLVVN

SEQ ID NO: 2 - protein, 140 aa, synthetic construct
AANAPGGSNVVNETPAQTVEVRAAPDALAFAQTSLSLPANTVVRLDFVNQNNLGVQHNWVLVNGGDDVAAAVNTAAQNNADALFVPPPDTPNALAWTAMLNAGESGSVTFRTPAPGTYLYICTFPGHYLAGMKGTLTVTP

SEQ ID NO: 3 - RNA, 23 nt, synthetic construct
  source 1..23: mol_type = other RNA
ctacctgcactataagcacttta

SEQ ID NO: 4 - protein, 29 aa, synthetic construct
CLSTAADMQGVVTDGMASGLDKDYLKPDD

SEQ ID NO: 5 - RNA, 21 nt, synthetic construct
  source 1..21: mol_type = other RNA
cagtacttttgtgtagtacaa

SEQ ID NO: 6 - RNA, 23 nt, synthetic construct
  source 1..23: mol_type = other RNA
ctacctgcactataagcacttta

SEQ ID NO: 7 - RNA, 54 nt, synthetic construct
  source 1..54: mol_type = other RNA
gtcgtatccagtgcagggtccgaggtcggcaattgcactggatacgacctacct

SEQ ID NO: 8 - DNA, 20 bp, synthetic construct
  source 1..20: mol_type = other DNA
gtaaagtgcttatagtgcag

SEQ ID NO: 9 - DNA, 16 bp, synthetic construct
  source 1..16: mol_type = other DNA
gtgcagggtccgaggt

SEQ ID NO: 10 - DNA, 50 bp, synthetic construct
  source 1..50: mol_type = other DNA
gtcgtatccagtgcagggtccgaggtattcgcactggatacgacctacct

SEQ ID NO: 11 - DNA, 21 bp, synthetic construct
  source 1..21: mol_type = other DNA
gtataccaaagtgcttacagt

SEQ ID NO: 12 - DNA, 49 bp, synthetic construct
  source 1..49: mol_type = other DNA
gtcgtatccagtgcagggtccgaggtattcgcactggatacgacctatt

SEQ ID NO: 13 - DNA, 21 bp, synthetic construct
  source 1..21: mol_type = other DNA
gtatactaaggtgcatctagt

SEQ ID NO: 14 - DNA, 50 bp, synthetic construct
  source 1..50: mol_type = other DNA
gtcgtatccagtgcagggtccgaggtattcgcactggatacgactgtagt

SEQ ID NO: 15 - DNA, 21 bp, synthetic construct
  source 1..21: mol_type = other DNA
gtatacagttttgcatagttg

SEQ ID NO: 16 - DNA, 50 bp, synthetic construct
  source 1..50: mol_type = other DNA
gtcgtatccagtgcagggtccgaggtattcgcactggatacgacgctgga

SEQ ID NO: 17 - DNA, 21 bp, synthetic construct
  source 1..21: mol_type = other DNA
gtatacagttttgcaggtttg

SEQ ID NO: 18 - DNA, 54 bp, synthetic construct
  source 1..54: mol_type = other DNA
gtcgtatccagtgcagggtccgaggtcggcaattgcactggatacgacctacct

SEQ ID NO: 19 - DNA, 20 bp, synthetic construct
  source 1..20: mol_type = other DNA
gtaaagtgcttatagtgcag

SEQ ID NO: 20 - DNA, 50 bp, synthetic construct
  source 1..50: mol_type = other DNA
gtcgtatccagtgcagggtccgaggtattcgcactggatacgacagcatt

SEQ ID NO: 21 - DNA, 21 bp, synthetic construct
  source 1..21: mol_type = other DNA
gtatacaggttgggatcggtt

SEQ ID NO: 22 - DNA, 50 bp, synthetic construct; identical to SEQ ID NO: 10
  source 1..50: mol_type = other DNA

SEQ ID NO: 23 - DNA, 21 bp, synthetic construct
  source 1..21: mol_type = other DNA
gtatacaaaagtgcttacagt

SEQ ID NO: 24 - DNA, 50 bp, synthetic construct
  source 1..50: mol_type = other DNA
gtcgtatccagtgcagggtccgaggtattcgcactggatacgactaaagt

SEQ ID NO: 25 - DNA, 21 bp, synthetic construct
  source 1..21: mol_type = other DNA
gtatacctacctgcactataa

SEQ ID NO: 26 - DNA, 56 bp, synthetic construct
  source 1..56: mol_type = other DNA
gtcgtatccagtgcgtgtcgtggagtcggcaattgcactggatacgacaaaatatg

SEQ ID NO: 27 - DNA, 17 bp, synthetic construct
  source 1..17: mol_type = other DNA
ttagcatggcccctgcg

SEQ ID NO: 28 - DNA, 16 bp, synthetic construct
  source 1..16: mol_type = other DNA
gtgcagggtccgaggt

SEQ ID NO: 29 - DNA, 19 bp, synthetic construct
  source 1..19: mol_type = other DNA
tacttgtggagccgctgac

SEQ ID NO: 30 - DNA, 20 bp, synthetic construct
  source 1..20: mol_type = other DNA
tgccagagaagagtgtggtg

SEQ ID NO: 31 - DNA, 20 bp, synthetic construct
  source 1..20: mol_type = other DNA
cggcgcatctatgacatcac

SEQ ID NO: 32 - DNA, 19 bp, synthetic construct
  source 1..19: mol_type = other DNA
gtcaacccctcaagccgtc

SEQ ID NO: 33 - DNA, 21 bp, synthetic construct
  source 1..21: mol_type = other DNA
catcatctcctggacctcacg

SEQ ID NO: 34 - DNA, 25 bp, synthetic construct
  source 1..25: mol_type = other DNA
acctcaggctggggtgggcagtctt

SEQ ID NO: 35 - DNA, 19 bp, synthetic construct
  source 1..19: mol_type = other DNA
cggaccgttctatgactgc

SEQ ID NO: 36 - DNA, 18 bp, synthetic construct
  source 1..18: mol_type = other DNA
agcgtgttctggctatgc

SEQ ID NO: 37 - DNA, 21 bp, synthetic construct
  source 1..21: mol_type = other DNA
cgggtttcaacgccgactacg

SEQ ID NO: 38 - DNA, 19 bp, synthetic construct
  source 1..19: mol_type = other DNA
gcaacgcagacttctcatc

SEQ ID NO: 39 - DNA, 20 bp, synthetic construct
  source 1..20: mol_type = other DNA
cggaactcttgtgcgtaagg

SEQ ID NO: 40 - DNA, 20 bp, synthetic construct
  source 1..20: mol_type = other DNA
ctcagccaaggttgtgaggt

SEQ ID NO: 41 - DNA, 18 bp, synthetic construct
  source 1..18: mol_type = other DNA
ggttgttcccaaatgctg

SEQ ID NO: 42 - DNA, 20 bp, synthetic construct
  source 1..20: mol_type = other DNA
ctccttaggtaagtcgtcca

SEQ ID NO: 43 - DNA, 19 bp, synthetic construct
  source 1..19: mol_type = other DNA
tggcgttaccttggtaacc

SEQ ID NO: 44 - DNA, 19 bp, synthetic construct
  source 1..19: mol_type = other DNA
ggtgttgagccctttccag

SEQ ID NO: 45 - DNA, 20 bp, synthetic construct
  source 1..20: mol_type = other DNA
agagtctataggcccacccc

SEQ ID NO: 46 - DNA, 20 bp, synthetic construct
  source 1..20: mol_type = other DNA
gctcgacgctaggatctgac

SEQ ID NO: 47 - DNA, 18 bp, synthetic construct
  source 1..18: mol_type = other DNA
aagaccatgtggacctgt

SEQ ID NO: 48 - DNA, 20 bp, synthetic construct
  source 1..20: mol_type = other DNA
ggtagaaatctgtcatgctg

SEQ ID NO: 49 - DNA, 20 bp, synthetic construct
  source 1..20: mol_type = other DNA
gatgtgatgcctctgcgaag

SEQ ID NO: 50 - DNA, 22 bp, synthetic construct
  source 1..22: mol_type = other DNA
catgctgatgtctctggaatct

SEQ ID NO: 51 - DNA, 18 bp, synthetic construct
  source 1..18: mol_type = other DNA
accataacccaccacagc

SEQ ID NO: 52 - DNA, 18 bp, synthetic construct
  source 1..18: mol_type = other DNA
cagttcgtccctttccag

SEQ ID NO: 53 - DNA, 20 bp, synthetic construct
  source 1..20: mol_type = other DNA
acggatttggtcgtattggg

SEQ ID NO: 54 - DNA, 20 bp, synthetic construct
  source 1..20: mol_type = other DNA
cgctcctggaagatggtgat

SEQ ID NO: 55 - DNA, 21 bp, synthetic construct
  source 1..21: mol_type = other DNA
aggtcggtgtgaacggatttg

SEQ ID NO: 56 - DNA, 19 bp, synthetic construct
  source 1..19: mol_type = other DNA
ggggtcgttgatggcaaca

SEQ ID NO: 57 - DNA, 36 bp, synthetic construct
  source 1..36: mol_type = other DNA
gtaaaacgacggccaacgcctgtaatcccagcactt

SEQ ID NO: 58 - DNA, 34 bp, synthetic construct
  source 1..34: mol_type = other DNA
gtaaaacgacggccatcgcccaggctggagtgca

SEQ ID NO: 59 - DNA, 37 bp, synthetic construct
  source 1..37: mol_type = other DNA
gtaaaacgacggccagtgtaaagtgcttatagtgcag

SEQ ID NO: 60 - DNA, 38 bp, synthetic construct
  source 1..38: mol_type = other DNA
gtaaaacgacggccagtgtatacctacctgcactataa

SEQ ID NO: 61 - DNA, 32 bp, synthetic construct
  source 1..32: mol_type = other DNA
aacagctatgaccatggtgcagggtccgaggt

SEQ ID NO: 62 - DNA, 14 bp, synthetic construct
  source 1..14: mol_type = other DNA
cgtttcatgaccac

SEQ ID NO: 63 - DNA, 14 bp, synthetic construct
  source 1..14: mol_type = other DNA
gtagcagggggatc

SEQ ID NO: 64 - protein, 28 aa, synthetic construct
LSTAADMQGVVTDGMASGLDKDYLKPDD

SEQ ID NO: 65 - protein, 28 aa, synthetic construct
XSXAADXXXVVXDXXASGLDKDYLKPDX

SEQ ID NO: 66 - protein, 28 aa, synthetic construct
XDPKLYDKDLGSAXXDXVVXXXDAAXSX

SEQ ID NO: 67 - protein, 128 aa, synthetic construct
AECSVDIQGNDQMQFNTNAITVDKSCKQFTVNLSHPGNLPKNVMGHNWVLSTAADMQGVVTDGMASGLDKDYLKPDDSRVIAHTKLIGSGEKDSVTFDVSKLKEGEQYMFFCTFPGHSALMKGTLTLK

(19) United States
(12) Patent Application Publication
(10) Pub. No.: US 2026/0193647 A1
(43) Pub. Date: Jul. 9, 2026

(54) MIRNA-BASED CANCER THERAPY WITH A TUMOR-NAVIGATING PEPTIDE

(71) Applicant: The Board of Trustees of the Universities of Illinois, Urbana, IL (US)

(72) Inventors: Sunam MANDER, Urbana, IL (US); Tapas K. DAS GUPTA, River Forest, IL (US); Tohru YAMADA, Oak Park, IL (US)

(21) Appl. No.: 18/868,924

(22) PCT Filed: May 26, 2023
(86) PCT No.: PCT/US2023/067542

§ 371 Date: Nov. 25, 2024

Related U.S. Application Data
(60) Provisional application No. 63/365,417, filed on May 27, 2022.

(51) Int. Cl. C12N 15/113; A61K 47/64; A61P 35/00; C12Q 1/6886

(52) U.S. Cl. CPC C12N 15/113; A61K 47/64; A61P 35/00; C12Q 1/6886; C12N 2310/11; C12N 2310/141; C12N 2310/3513; C12Q 2600/178

(57) ABSTRACT

A nanomedicine platform that involves the use of a blood brain-permeable tumor-navigating probe that includes a peptide p28 covalently linked with antisense miRNA. The probe localized in intracerebral human pediatric glioblastoma tumors in mice. Upon cellular entry, the probe significantly inhibits tumor cell viability by silencing the oncogenic miRNA miR-20a. Notably, systemic administration of the probe enabled complete regression of the ear-ly-stage tumor and significantly prolonged overall survival without apparent adverse effects.

### CROSS-REFERENCE TO RELATED APPLICATIONS

This application claims the benefit of priority to U.S. provisional patent application No. 63/365,417, filed on May 27, 2022, which is incorporated by reference herein in its entirety.

### STATEMENT AS TO FEDERALLY SPONSORED RESEARCH

This invention was made with Government support under Federal Grant Nos. R21CA252370 awarded by NIH/NCI R21. The Government has certain rights to this invention.

### REFERENCE TO A SEQUENCE LISTING

The instant application contains a Sequence Listing which has been filed electronically in ASCII format and is hereby incorporated by reference in its entirety. Said ASCII copy, created on May 26, 2023, is named 46466-59.xml and is 61440 bytes in size.

### BACKGROUND OF THE INVENTION

Glioblastoma is the most common primary malignant brain tumor; however, glioblastoma is less common in children than in adults, and pediatric glioblastoma (pGBM) remains a devastating disease with substantial morbidity and mortality. pGBM has a median survival rate from 13 to 73 months, with a 5-year survival rate of less than 20%. pGBM treatment currently involves mainly gross total resection followed by local irradiation with additional chemotherapy. Despite these treatments, pGBM remains incurable. The major challenges in the development of new therapeutic agents are i) limited transport through the blood-brain barrier (BBB), ii) poor intracellular penetration, and iii) tumor heterogeneity. In addition, clinical trials for children with glioma are often based on regimens that do not account for differences in tumor biology between children and adults. In recent years, our understanding of the origin and biological features of pediatric brain tumors has substantially increased through genome and epigenome molecular profiling. These results have shown that aberrant expression of microRNA (miRNA) is critical in cancer progression.

miRNAs are small (18-25 nucleotides) noncoding RNA molecules. Unlike small interfering RNA (siRNA), a single miRNA can regulate multiple target genes, thereby simultaneously regulating multiple signaling pathways, including proliferation, angiogenesis, and differentiation.

Although using antisense-miRNA to diminish overexpressing oncogenic miRNA is a promising strategy, as it selectively targets and silences ‘druggable’ and ‘undruggable’ genes, the effective and safe delivery of antisense miRNA to target tissues remains a major challenge for miRNA-based therapies. To develop an effective delivery system for miRNA, biocompatible non-viral based miRNA delivery agents, such as cell-penetrating peptides (CPPs), lipids, and extracellular vesicle carriers have been explored. Among these delivery systems, CPPs are considered as a promising strategy to improve intracellular delivery and the versatile nature to combine with other delivery vehicles to enhance cell or tissue penetrating ability. More importantly, for targeting pGBM, the presence of the BBB limits penetration of the vehicles into the tumor site, reducing the delivery and thus efficiency of miRNA-based agents. Therefore, the development of effective carriers that can overcome these challenges is necessary, and new miRNA-based therapeutic approaches remain an unmet medical need in the absence of alternative therapeutic approaches for pGBM.

Therapeutic approaches have been developed to either suppress or restore the expression of miRNAs associated with the disease such as virus-based miRNA and anti-miRNA oligonucleotide delivery systems. However, the safe and efficient delivery of miRNAs to target tissues remains a significant challenge for miRNA-based therapies. To overcome the challenges, less toxic and biocompatible non-viral based miRNA delivery such as cell penetrating peptides (CPPs), lipid, and extracellular vesicle carrier are explored. Among these delivery systems, CPPs are considered as a promising strategy to improve intracellular delivery due to their simplicity of chemical and biological synthesis, efficient tissue penetration owing to their small hydrodynamic sizes, and the versatile nature to combine with other delivery vehicles to enhance cell or tissue penetrating ability.

In addition, the complexity of brain and the presence of physiological Blood Brain Barrier (BBB) limits penetration of majority of molecules into the brain/tumor site, reducing the efficiency of oligonucleotide drugs. Therefore, the systematic design of vectors that can overcome these delivery challenges is necessary for the successful targeted delivery of miRNA into the tumor site of the brain.

### SUMMARY OF THE INVENTION

We previously developed p28, a CPP derived from Pseudomonas aeruginosa azurin that preferentially enters cancer cells and inhibits proliferation. p28 (NSC745104) as a single therapeutic agent was clinically tested in two phase I clinical trials and granted FDA Orphan Drug Designation for the treatment of pediatric high-grade gliomas and Rare Pediatric Disease Designation for the treatment of diffuse intrinsic pontine glioma, as p28 was well tolerated without apparent adverse effects, toxicity, or immunogenicity in pediatric patients with recurrent and refractory central nervous system tumors (NCI and Pediatric Brain Tumor Consortium) and in adult patients with advanced solid tumors. Two phase I clinical trials have demonstrated the safety and non-toxic properties of p28, but its efficacy was moderate. To improve the efficacy, we used p28 as a carrier molecule since it has great potential for an effective and safe tumor-targeted approach.

We designed a new and unique agent, AmiR20-p28 by covalently linking the BBB-permeable tumor-navigating CPP p28 and antisense miR-20a to target miR-20a, which is specifically and highly overexpressed in pGBM over aGBM and normal brain cells. After systemic administration, AmiR20-p28 crosses the BBB, preferentially accumulates in pGBM tumors, and efficiently inhibits oncogenic miR-20a expression. Further experiments in a pGBM mouse model generated with drug-resistant cancer stem cells showed that AmiR20-p28 reduced the tumor burden and significantly prolonged overall survival without apparent adverse effects. Our findings develop a nanomedicine platform that involves the use of the BBB-permeable tumor-navigating peptide p28 linked with antisense miRNAs. It offers a broadly applicable strategy for cancer treatment with potential for clinical translation.

Other methods, features and/or advantages is, or will become, apparent upon examination of the following figures and detailed description. It is intended that all such additional methods, features, and advantages be included within this description and be protected by the accompanying claims.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-1B: FIG. 1A: graphic representing Mean-difference plot showing the log 2-fold change (log 2FC) and expression of each gene (log 2Exp) between pGBM and normal brain. Dark Grey: significantly (p<0.05) upregulated, Light Grey: significantly downregulated. FIG. 1B: qPCR analysis showing expression level of miR17-92 clusters in SJ-GBM2, CHLA-200, LN-229 and Astrocytes cell lines. Values are expressed as mean±SEM. Statistical significance: **p<0.01 and ***p<0.001.

FIG. 2A-2G: Overexpression of miR-20a in pGBM. FIG. 2A, The miR-17-92 cluster analyses in pGBM cell lines. The y-axis indicates the -log 10 of the p values and the x-axis is the fold change of the expression between different cell lines as compared to astrocytes. Circle: SJ-GBM2, square: CHLA-200, triangle: LN-229. FIG. 2B, The miR-17-92 cluster analyses in pediatric patients with anaplastic astrocytoma (square, ID #4) and malignant glioma (circle, ID #5). The y-axis indicates the -log 10 of the p values and the x-axis is the fold change of the expression between different cell lines. FIG. 2C, qPCR analysis showing expression level of miR20a in different cancer cell lines: SJ-GBM2, CHLA-200, LN-229, MDA-MB231, SKOV3, MiaPaca2, Colon 205, DU145, and Mel 2. The y-axis represents relative expression level normalized to U6 (2^{-ΔΔCt} value). The values are expressed as mean±SEM. **p<0.01 and ****p<0.0001. FIG. 2D, Preferential penetration of AmiR20-p28, confocal microscopy analysis of the p28 uptake by SJ-GBM2, CHLA-200 and astrocytes cells. Cells were treated with 20 μM of Alexa Fluor labeled peptide for 2 hr at 37° C. and subjected to confocal analysis. Alexa fluor dye-p28 (red in color); Nucleus (blue in color). FIG. 2E, SJ-GBM2, CHLA-200 and astrocytes were treated with 20 μM of Alexa Fluor labeled AmiR20-p28 for 2 hr at 37° C. Red: Alexa fluor dye-AmiR20-p28; Blue: Nucleus. FIG. 2F, The Ct values of anti-sense miR20 obtained from treatment of SJ-GBM2, CHLA-200, LN-229 and astrocytes with AmiR20-p28 at 1, 10, 100 and 1,000 nM for 24 hr. MicroRNA was isolated with miRNeasy and antisense-miR20 was detected by stem loop qPCR. FIG. 2G, An agarose gel image with the end products of qPCR from SJ-GBM2, CHLA-200, LN-229 and astrocytes subjected to 1 nM AmiR20-p28.

FIG. 3A-3C: Preferential p28 entry. FIG. 3A, Confocal images of p28 uptake by SJ-GBM2, CHLA-200 and astrocytes cells. Cells were treated with 20 μM of Alexa Fluor labeled p28 peptide for 2 hr at 37° C. and images were recorded. Red: Alexa fluor dye-p28; Blue: Nucleus. FIG. 3B-3C: Chemical reactions of the maleimide-thiol conjugation between 5′-Maleimide antisense-miR20a and Cys-p28 (FIG. 3B), and click chemistry conjugation of hexynyl-antisense miR20 and azide-p28 (FIG. 3C).

FIG. 4A-4E: Anti-proliferative effect of AmiR20-p28 by silencing miR-20a in pGBM cells. FIG. 4A, Expression levels of miR-20a in SJ-GBM2, CHLA-200, LN-229 and astrocytes that were treated with 10 nM p28, AmiR20, negative control (Neg Con), or AmiR20-p28 for 24 hr. FIG. 4B, Cell viability was determined by the CCK-8 assay. SJ-GBM2, CHLA-200, LN-229 and astrocytes were treated with p28, AmiR20, Neg Con, or AmiR20-p28 for 24 hr. FIG. 4C, Flow cytometric analyses of apoptotic cells treated with 10 nM p28 or AmiR20 or AmiR20-p28 for 24 hr. Each cell line was processed with Annexin-V/PI staining. The values are expressed as mean±SEM. NS: not significant. *p<0.05, ***p<0.001, ****p<0.0001.

FIG. 4D, pGBM (SJ-GBM2 and CHLA-200) was exposed to 10 nM p28 or AmiR20 or AmiR20-p28 for 24 hr and subsequently assessed for gene expressions by qPCR. Mean±SEM. NT (control) was expressed as 1.0. *p<0.05, **p<0.01, ***p<0.001, ****p<0.0001. FIG. 4E, Western blots of major proteins in the signaling pathways. Each cell line was treated with 10 nM p28 or AmiR20 or AmiR20-p28 for 24 hr, and whole cell lysates were separate by gel electrophoresis. Actin was used as an internal loading control.

FIG. 5A-5C: Effect of AmiR20-p28. FIG. 5A, SJ-GBM2, CHLA-200, LN-229 and normal human astrocytes were exposed to 10 nM p28, AmiR20 or AmiR20-p28, prepared from click chemistry conjugation. Cell viability was evaluated by the CCK-8 assay after 24 hr of incubation. Mean±SEM. ****p<0.0001 (ANOVA). FIG. 5B, Annexin-V/FITC/PI flow cytometry analysis of SJ-GBM2, CHLA-200, LN-229 and astrocytes treated with 10 nM p28, AmiR20 or AmiR20-p28. The viable, early apoptotic, late apoptotic and necrotic cells are represented by the lower left quadrant (Annexin-V −/PI−), lower right (Annexin-V +/PI−), upper right (Annexin-V +/PI+) and upper left (Annexin-V −/PI+), respectively. FIG. 5C, MDA-MB231 (breast cancer), SKOV3 (ovarian cancer), Mia-Paca2 (pancreatic cancer), Colon 205 (colon cancer), DU145 (prostate cancer) and Mel 2 (melanoma) cells were treated with 10 nM p28, AmiR20 or AmiR20-p28 and subjected to a CCK-8 assay.

FIG. 6A-6C: Modulation of gene expressions by AmiR20-p28. FIG. 6A-6B LN-229 (FIG. 6A) and astrocyte (FIG. 6B) were treated with 10 nM p28, AmiR20 or AmiR20-p28 for 24 hr and subsequently assessed for gene expression analyses. NT (control) was expressed as 1.0. Mean±SEM. **p<0.01, ***p<0.001 (ANOVA). FIG. 6C, Proposed signaling pathways modulated by AmiR20-p28. Blue arrows represent the downregulation of the associated gene whereas red arrows represent the upregulation of the associated gene by AmiR20-p28.

FIG. 7A-7D: AmiR20-p28 exhibits cytotoxic activity in SJ-GBM2 having glioma stem cells (GSC) phenotype. FIG. 7A, The IC₅₀ values of TMZ in pGBM cells (SJ-GBM2 and CHLA-200). Each cell line was exposed to TMZ for 24 hr, and cell viability was measured by CCK-8 assay. *p<0.05 (SJ-GBM2 vs CHLA-200, t-test). FIG. 7B, pGBM cells (SJ-GBM2 and CHLA-200) and normal human astrocytes were stained for the GSC marker CD133 (green, in color). The cells were also treated with Alexa Fluor-labeled AmiR20-p28 (red in color). DAPI (in color). FIG. 7C, Confocal images showing AmiR20-p28 uptake by SJ-GBM2 cell spheroids. The spheroids were treated with 20 μM Alexa Fluor-labeled AmiR20-p28 for 2 hr at 37° C. and analyzed by confocal microscopy. Alexa Fluor-AmiR20-p28 (red in color); nucleus (blue in color). FIG. 7D, Representative images of spheroids treated with or without 10 nM AmiR20-p28 for 24 and 48 h (arrowheads: disintegrated structure of the SJ-GBM2 spheroids).

FIG. 8A-8J: AmiR20-p28 crosses the BBB, inhibits tumor growth, and enhances overall survival. FIG. 8A, T₂-weighted MR images (top left: coronal, bottom left: sagittal) and H&E-stained sections (bottom right) of a mouse brain after injection with SJ-GBM2 cells. NIR (near infrared fluorescence) brain image from the mice that received ICG-labeled p28 at 0.5 mg/kg (top right: light photo, middle right: NIR image at 800 nm). FIG. 8B, Illustrative image of the 3D BBB assay. Bottom left: BBB permeability assay for AmiR20-p28 and antisense miR-20 (apical to basolateral). Bottom right: The influx (blood to brain), apical to basolateral (A to B), efflux (brain to blood), and basolateral to apical (B to A) transport of AmiR20-p28 was determined in the BBB model. ***p<0.001, ****p<0.0001 (t test). FIG. 8C, Quantification of the luciferase signal in the ROI from SJ-GBM2-luc cells in mice treated with PBS or 1 or 5 mg/kg AmiR20-p28 i.v. (N=5 in each group). At day 28 (arrow), the groups receiving AmiR20-p28 at 1 and 5 mg/kg were further divided into two subgroups: one group received continued treatment for additional ~two months (3× weekly i.v, N=2 in each dose), while the other group (N=3 in each dose) stopped treatment to see if tumors recurred. Representative bioluminescent images of tumor growth at day 63. ****p<0.0001 (PBS vs all treatment groups at day 63, ANOVA). FIG. 8D, A schematic image of the experimental design (top). Quantification of bioluminescence in the ROI from SJ-GBM2-luc tumors. SJ-GBM2 xenograft mice received 1 mg/kg AmiR20-p28 i.v. at three different tumor growth phases [at 10 (early phase), 20 (middle phase), and 40 (late phase) days after cell injection]. Injections of PBS and AmiR20 (antisense miR-20a alone) were initiated at 10 days after cell injection. N=5 in each group *p<0.05 (late-phase vs. mid-phase, ANOVA), ****p<0.0001 (late-phase vs. early-phase ANOVA). FIG. 8E, Representative images showing merged bioluminescence from SJ-GBM2-luc tumors and X-ray images of mice from the early-phase (upper) and late-phase (lower) groups. FIG. 8F, Kaplan-Meier survival curve of SJ-GBM2 xenograft mice. The p values shown were obtained using the log-rank (Mantel-Cox) test. FIG. 8G, Detection of AmiR20-p28 in SJ-GBM2 tumors. Mice with SJ-GBM2-luc received 1 mg/kg AmiR20-p28. After 30 and 120 min, miRNA was extracted from tumor tissues and normal brain tissues, and antisense miR-20 was detected by real-time PCR. PCR products were visualized on a 2% agarose gel. FIG. 8H, Expression of miR-20a in normal brain tissues and tumors obtained from the mice treated with PBS or 1 mg/kg AmiR20-p28 3 times a week for one week. ***p<0.001 (ANOVA). FIG. 8I, Circulating miR-20a levels in sera from healthy human volunteers (N=5), pediatric patients with GBM (ID #16) and malignant glioma (ID #17). miRNA-enriched fractions were obtained by miRNeasy kits, and circulating miR-20a levels were determined by qPCR. ****p<0.0001 (two-tailed t-test). FIG. 8J, Circulating miR-20a levels were measured in sera from the tumor-free mice and the mice treated with PBS or (early, mid-, or late-phase) AmiR20-p28. ***p<0.001, ****p<0.0001 (vs. sera from tumor-free mice), p=0.78 (tumor-free mice vs. early-phase treatment with AmiR20-p28), ###p<0.001 (PBS vs. early-phase treatment with AmiR20-p28).

FIG. 9A-9C: Tumor preferential localization of p28. FIG. 9A, Coronal and sagittal sections of T₂-weighted MR images and NIR images of the brain tumor. SJ-GBM2 tumor development was confirmed by MRI (yellow-arrowheads). NIR fluorescence of dorsal and coronal (yellow dotted lines) brain images were taken 24 hr post i.v injection with 0.5 mg/kg ICG-p28. FIG. 9B, Coronal and sagittal sections of MR images of tumor free mice brain as negative control images. NIR images were taken 24 hr post i.v injection with 0.5 mg/kg ICG-p28. Both MRI and NIR images showed no specific signal. FIG. 9C, Coronal and sagittal section of T₂-weighted MR images of mice bearing orthotopic brain tumor. SJ-GBM2 tumor development was confirmed by MRI. NIR images were taken 24 hr post i.v injection with 0.5 mg/kg ICG dye alone. These showed that the ability of tumor preferential localization of ICG-p28 was due to the p28 motif.

FIG. 10: AmiR20-p28 inhibits tumor growth in orthotopic pGBM. SJ-GBM2-luc cells were implanted intracranially. ROI quantification of luciferase signals from SJ-GBM2-luc cells in mice treated with PBS, 0.1 or 1 mg/kg AmiR20-p28 i.v. 3× weekly. N=5 in each group. Representative bioluminescent images of each group. Mean±SEM. ***p<0.001 (ANOVA).

FIG. 11A-11B: AmiR20-p28 treatment at early-phase tumor development completely represses tumor growth in orthotopic pGBM. FIG. 11A, Time-course of bioluminescent images of tumor progression in mice treated with PBS, 1 mg/kg AmiR20, 1 mg/kg AmiR20-p28 i.v. (early-, mid- or late-phase groups). Red-X indicates mouse dead or eliminated based on the humane endpoints criteria. FIG. 11B, H&E staining of the brain section from mice in the early-phase treatment group. There was no tumor residues confirmed at the injection site.

FIG. 12: Clinical signs reflect the anti-tumor efficacy of AmiR20-p28. The representative mouse at the late-phase shows moribund body state whereas normal behavior of the early-phase mouse.

FIG. 13A-13D: FIG. 13A-13B, Tumor free Balb/c mice (4 weeks old) received 5× and 10× higher dose of the optimum AmiR20-p28 (1 mg/kg) i.v. After 24 hr of the single-dose injection, ALT (FIG. 13A) and AST (FIG. 13B) were measured and compared to negative control (PBS) and positive control (acetaminophen) animals. ****p<0.0001 (ANOVA). FIG. 13C, Hemolysis assay showing percentage of hemolysis of erythrocytes exposed to 1, 10, 100, 1,000, 10,000 nM AmiR20-p28. Triton X-100 detergent was used as a positive control. NS: not significant (vs. negative control at 0 nM AmiR20-p28). FIG. 13D, Histological evaluation of AmiR20-p28 in major organs. Mice from all the treatment group (FIG. 8D) were evaluated. Representative H&E-stained heart, lung, liver, spleen, and kidney showed that there was no AmiR20-p28-related differences in histology. Scale bars, 200 μm.

FIG. 14A-14B: Isolation of pGBM from normal brain tissues. FIG. 14A, Bioluminescence merged to X-ray image showed SJ-GBM2-luc tumor location in the brain (left). Following the conformation of signal from tumor with IVIS, brain tumor was resected and separated from normal brain tissues. FIG. 14B, To confirm the brain tumor isolation, the PCR-based method was also applied. Quantitative analyses of hAlu DNA and mGAPDH DNA to detect the human DNA in mouse tissue sample confirmed clear isolation of brain tumor from normal brain tissues. Mean±SEM. ****p<0.0001 (two-tailed t-test). The PCR products were visualized by agarose gels.

FIG. 15: Circulating miR-20a in mice sera correlates to tumor growth inhibition. Circulating miR-20a levels in mice sera (FIG. 8J) and bioluminescence from tumor at day 70 (FIG. 8D) were plotted.

FIG. 16: Estimation of xenograft tumor size in humans. Conversion of ROI to cancer cell numbers by using a standard curve of linear regression. Bioluminescence from various numbers (10²-10⁷ cells) of SJ-GBM2-luc was determined by the Lago X imaging system. The cell number for desired luminescent signal from ROI was measured and converted into relative tumor size in pediatric brain. The averages of mice brain size (0.58 cm³) and pediatric brain size (1,312 cm³) were used.

FIG. 17A-17E: Antitumor activity of AmiR20-p28 in orthotopic xenografts using SJ-GBM2-luc cells. FIG. 17A, Left: ROI quantification of luciferase signal from SJ-GBM2-luc cells in mice. Right: Representative bioluminescent images of tumor growth in mice treated with AmiR20 or AiR20-p28. PBS, n=5; AmiR20, n=5; AmiR20-p28, n=5. FIG. 17B, Top: Experimental design. Left: ROI quantification of SJ-GBM2-luc cells tumor radiance. PBS, n=5; AmiR20, n=5; AmiR20-p28 (early), n=5; AmiR20-p28 (mid), n=5; AmiR20-p28 (late), n=5. Right: Representative IVIS images of luciferase signal in mice receiving 1 mg/kg AmiR20-p28 (top: late phase, middle: mid phase, bottom: early phase). FIG. 17C, Kaplan-Meier survival curve of SJ-GBM2 xenografts mice who received 1 mg/kg AmiR20-p28 treatment. FIG. 17D, Detection of AmiR20-p28 in tumor specific regions. Mice were treated with 1 mg/kg AmiR20-p28 for 30 and 120 min followed by the extraction of microRNA from the tumor and normal brain tissue regions. Visualization of end products of real time PCR in 2% agarose gel.

FIG. 17E, Confocal images of brain sections. Mice bearing SJGBM2 tumors were injected Alexa fluor 568 labeled AmiR20-p28. Cryosection of the brain sections were imaged.

FIG. 18A-18E: Adult glioblastomas (aGBM) LN229 showed the highest levels of miR21 compared to pGBM (FIG. 18A). Ten nM AmiR21-p28 exposure to pGBM and aGBM significantly reduced the endogenous miR21 (FIGS. 18B-18D), but not in human normal astrocytes (FIG. 18E).

FIG. 19A-19D: Alternations of gene expression profiles induced by AmiR21-p28 are different from those induced by AmiR20-p28.

### DETAILED DESCRIPTION OF THE INVENTION

Although pGBM is histologically indistinguishable from adult glioblastoma (aGBM), significant differences occur at the molecular level. Analysis of miRNA expression profiles in aGBM showed significant alterations in the expression of a group of miRNAs, including miR-21, which is overexpressed in aGBM. In contrast, the expression of miRNA in the miR-17-92 cluster (miR-17, miR-18a, miR-19a, miR-19b-1, miR-20a, and miR-92a-1) was shown to be greatly elevated in pGBM compared to aGBM and healthy individuals.

Pediatric glioblastoma (pGBM) remains a devastating disease, as there is no standard curative chemotherapy regimen, partly due to poor cellular penetration, the lack of tumor targeting, and limited drug delivery through the blood-brain barrier (BBB). Therefore, we aim to develop a nanomedicine platform that involves the use of the BBB-permeable tumor-navigating peptide p28 covalently linked with antisense miRNA20a (namely, AmiR20-p28). Here, we show that AmiR20-p28 crossed the BBB and preferentially localized in intracerebral human pGBM tumors in mice. Upon cellular entry, AmiR20-p28 significantly inhibits pGBM cell viability by silencing the oncogenic miRNA miR-20a, which alters multiple signaling pathways. Notably, systemic administration of AmiR20-p28 enabled complete regression of the early-stage tumor and significantly prolonged overall survival without apparent adverse effects in orthotopically xenografted mice. Thus the development of a miRNA-based platform with p28 represents a translatable strategy for the treatment of pGBM.

### Definitions

As used in the specification and the claims, the singular forms “a,” “an,” and “the” include plural referents unless the context clearly dictates otherwise.

As used herein, the term “cell” includes either the singular or the plural of the term. The terms “isolated”, “purified” or “biologically pure” refer to material which is substantially or essentially free from components which normally accompany material as it is found in its native state. The term “heterologous DNA,” “heterologous nucleic acid sequence,” or “exogenous” and the like as used herein refers to a nucleic acid sequence wherein at least one of the following is true: (a) the sequence of nucleic acids foreign to (i.e., not naturally found in) a given host microorganism; (b) the sequence may be naturally found in a given host microorganism, but in an unnatural (e.g., greater than expected) amount; or (c) the sequence of nucleic acids comprises two or more subsequences that are not found in the same relationship to each other in nature.

“Peptide” and “polypeptide” are used interchangeably herein and refer to a compound made up of a chain of amino acid residues linked by peptide bonds. An “active portion” of a polypeptide means a peptide that is less than the full length polypeptide, but which retains measurable biological activity and retains biological detection.

As used herein, the term “tumor” refers to any neoplastic growth, proliferation or cell mass whether benign or malignant (cancerous), whether a primary site lesion or metastases.

As used herein “therapeutically effective amount” refers to an amount of a composition that relieves (to some extent, as judged by a skilled medical practitioner) one or more symptoms of the disease or condition in a mammal. Additionally, by “therapeutically effective amount” of a composition is meant an amount that returns to normal, either partially or completely, physiological or biochemical parameters associated with or causative of a disease or condition. A clinician skilled in the art can determine the therapeutically effective amount of a composition in order to treat or prevent a particular disease condition, or disorder when it is administered, such, as intravenously, subcutaneously, intraperitoneally, orally, or through inhalation. The precise amount of the composition required to be therapeutically effective will depend upon numerous factors, e.g., such as the specific activity of the active agent, the delivery device employed, physical characteristics of the agent, purpose for the administration, in addition to many patient specific considerations. But a determination of a therapeutically effective amount is within the skill of an ordinarily skilled clinician upon the appreciation of the disclosure set forth herein.

Treat”, “treating”, and “treatment”, etc., as used herein, refer to any action providing a benefit to a patient at risk for or afflicted with a disease, including improvement in the condition through lessening or suppression of at least one symptom, delay in progression of the disease, prevention or delay in the onset of the disease, etc. Treatment also includes partial or total destruction of the undesirable proliferating cells with minimal destructive effects on normal cells. A subject at risk is a subject who has been determined to have an above-average risk that a subject will develop cancer, which can be determined, for example, through family history or the detection of genes causing a predisposition to developing cancer.

The term “subject,” as used herein, refers to a species of mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos.

Where methods and steps described herein indicate certain events occurring in certain order, those of ordinary skill in the art will recognize that the ordering of certain steps may be modified and that such modifications are in accordance with the variations of the invention. Additionally, certain steps may be performed concurrently in a parallel process when possible, as well as performed sequentially.

The meaning of abbreviations is as follows: “C” means Celsius or degrees Celsius, as is clear from its usage, “s” means second(s), “min” means minute(s), “h,” “hr,” or “hrs” means hour(s), “psi” means pounds per square inch, “nm” means nanometers, “d” means day(s), “μL” or “uL” or “ul” means microliter(s), “mL” means milliliter(s), “L” means liter(s), “mm” means millimeter(s), “nm” means nanometers, “mM” means millimolar, “μM” or “uM” means micromolar, “M” means molar, “mmol” means millimole(s), “μmol” or “uMol” means micromole(s), “g” means gram(s), “μg” or “ug” means microgram(s) and “ng” means nanogram(s), “PCR” means polymerase chain reaction, “kDa” means kilodaltons, “g” means the gravitation constant, “bp” means base pair(s), “kbp” means kilobase pair(s), “% w/v” means weight/volume percent, “% v/v” means volume/volume percent, “rpm” means revolutions per minute, “HPLC” means high performance liquid chromatography, and “GC” means gas chromatography.

It is understood that “aurA”, “aurB”, “azurin” may refer to any molecule having a peptide sequence with substantial similarity to SEQ ID NOs: 1, 2, or 67 respectively in whole or part. It is appreciated that any polypeptide that includes partially SEQ ID NOs: 1, 2, or 67 and is approximately between 24-32 nucleotides in length may be considered 96% identical to p28 (SEQ ID NO: 64) and is usable as a probe in the methods described herein. That is, if a single amino acid of the aurA, aurB, or azurin partial sequence that is approximately 28 amino acids long (e.g. +3 amino acids) p28 is altered via substitution with a different amino acid, that new sequence would be 96% identical to SEQ ID NOs: 1, 2, or 67. Likewise is a single amino acid is added to the p28 sequence, would result in a sequence that is 96% identical to SEQ ID NOs: 1, 2, or 64. SEQ ID NOs 65 and 66 are exemplary sequences to describe the substantially similarity described herein. The modified cupredoxin derived peptide may comprise X₁SX₂AADX₃X₄X₅VVX₆DX₇X₈ASGLDKDYLKPDX₉ (SEQ ID NO: 65), where X₁ is L or acetylated-L, X₂ is T or W, X₃ is M, L or V, X₄ is Q or W, X₅ is G or A, X₆ is T or W, X₇ is G, T or W, X₈ is M, L or V, and X₉ is D or amidated-D. In other embodiments, the modified cupredoxin derived peptide may consist of X₁SX₂AADX₃X₄X₅VVX₆DX₇X₈ASGLDKDYLKPDX₉ (SEQ ID NO: 66), where X₁ is L or acetylated-L, X₂ is T or W, X₃ is M, L or V, X₄ is Q or W, X₅ is G or A, X₆ is T or W, X₇ is G, T or W, X₈ is M, L or V, and X₉ is D or amidated-D.

### Overview

Large datasets analyses revealed significant differences between pGBM and normal brain tissues at the molecular levels such as miRNA expression. In particular, miRNA20 is elevated in pGBM and functions as an oncogenic miRNA. As such, targeting specific oncogenic miRNA in pGBM is an attractive approach to develop new miRNA-based agents for pGBM. To diminish levels of oncogenic miRNA in pGBM by using antisense-miRNA is a promising approach, however, i) transport to the brain by crossing the blood-brain barrier (BBB), and ii) tumor-targeted delivery of anti-miRNAs are currently major challenges for advancement into the clinic. In this study, we overcome current limitations by using a cell penetrating-peptide, p28 as a carrier molecule. We have demonstrated that the redox protein azurin, secreted by an opportunistic pathogen Pseudomonas aeruginosa, preferentially enters human cancer cells and induces apoptosis. We have further identified that a fragment of azurin, p28, can cross the BBB and preferentially enters human pGBM cells, SJ-GBM2 and CHLA-200. In addition, p28 has no apparent toxicity or immunogenicity in a clinical trial of pediatric CNS patients. Thus, p28 is a potentially ideal carrier for the pGBM targeted delivery. Here, we created a unique agent by covalently linking p28 and antisense-miRNA20 (namely AmiR20-p28). AmiR20-p28 exposure showed preferentially penetration and clear dose-dependent anti-proliferative/apoptotic effects on pGBM cell lines, but not on human astrocytes used as a non-malignant control. In contrast, antisense-miRNA20 alone without p28 conjugation showed very little effect at any concentration tested in these cancer cells. Such apoptotic effects induced by AmiR20-p28 were associated with a significant reduction of endogenous oncogenic miRNA20 levels in pGBM.

Further, the p53 and ERK signaling pathways were altered by AmiR20-p28, suggesting that AmiR20-p28 can regulate multiple pathways simultaneously. Remarkably, AmiR20-p28 treatment at the early phase of pGBM tumor development enables complete regression and dramatically prolong survival in orthotopic xenograft mouse models with drug-resistant cancer-stem pGBM cells. The PCR analyses showed the preferential accumulation of AmiR20-p28 at the given tumor in the mouse brain. Based on these findings, the development of p28 as a carrier provides a novel strategy for tumor-targeted delivery of miRNAs. It can overcome the current limitations of developing miRNA-based agents and provide a positive impact on the treatment outcomes (e.g., morbidity, quality of life) for pGBM.

In some aspects, a composition that includes an antisense microRNA covalently linked to a cell penetrating peptide are described. The cell penetrating peptide may include p28, may include p28 as part of a larger peptide, may include only p28, may include a peptide with significant homology to p28, may include a peptide with a single amino acid variant from p28, or may include a peptide with 90% sequence identity to p28. The antisense microRNA is an antisense miR20, an antisense miR21, or an antisense miR21a or the antisense microRNA corresponding to any appropriate microRNA target. The antisense microRNA that is covalently linked to a cell penetrating peptide may be in any form desired for a pharmaceutical use. The covalent linkage between the microRNA and p28 may occur through a maleimide-thiol reaction. In some aspects, the covalent linkage between the microRNA and p28 may occur between a hexynyl-antisense microRNA and an azide-p28 through click chemistry conjugation.

In some aspects, a method of inhibiting tumor cell growth are described. Briefly, systemic administration to tumor cell of a composition comprising any of the compositions according to any of the aspects of the invention will result in inhibition of tumor cell growth.

In some aspects, a method of targeting an antisense microRNA to a tumor cell are described. Briefly, systemic administration to tumor cell of a composition comprising any of the compositions according to any of the aspects of the invention will permit targeting of antisense miRNA to and entry into a tumor cell. In some aspects, the cell is an in vivo cell. In some aspects, the antisense microRNA covalently linked to a cell penetrating peptide forms a complex that crosses the blood-brain-barrier.

In some aspects, a method of reducing endogenous microRNA in a tumor cell are described. Briefly, systemic administration to the tumor cell of any of the compositions according to any of the aspects of the invention results in a reduction of endogenous miRNA within a tumor cell. In some aspects, the tumor cell is an in vivo cell.

### EXAMPLES

### Example 1: MiR-20a is Expressed More Highly in pGBM than in aGBM and Normal Astrocytes

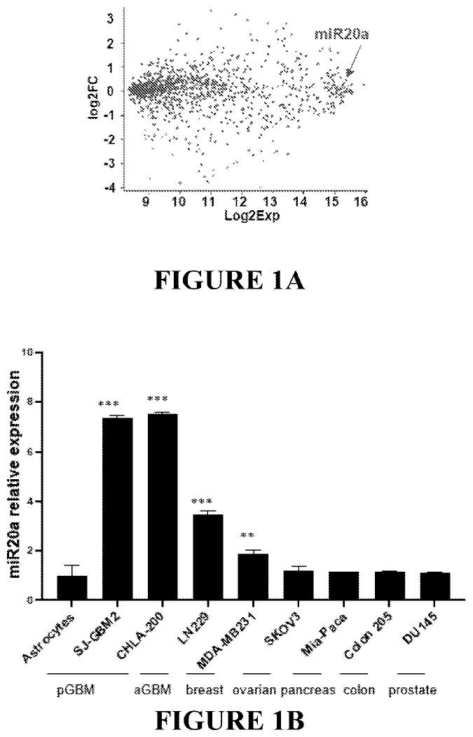

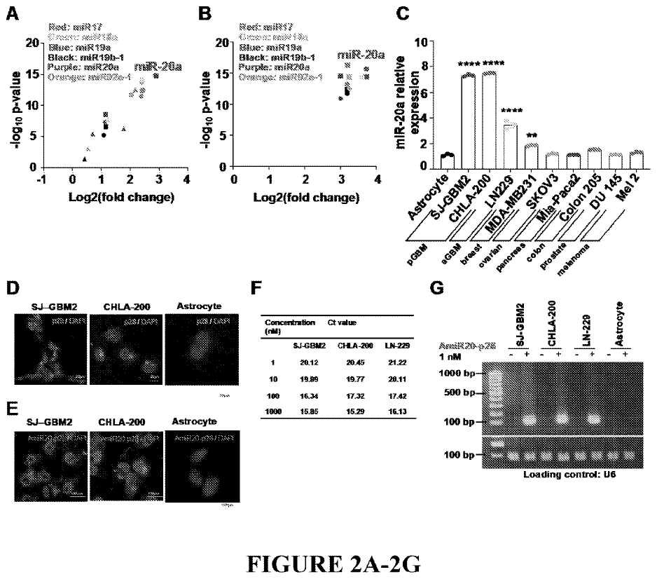

Reported gene expression profiles of the miR-17-92 cluster and their role in tumorigenic signaling in glioma prompted us to further investigate the role of this cluster in pGBM. In addition, miRNA profiling with the GSE42657 dataset confirmed that the miR-17-92 cluster genes aberrantly expressed in pGBM. In particular, miR-20a, a member of the miR-17-92 cluster, is significantly and highly expressed in pGBM (FIG. 1A). Therefore, we further investigated the differentially expressed miR-17-92 cluster genes in pGBM cell lines (SJ-GBM2 and CHLA-200), an aGBM cell line (LN-229), and normal human astrocytes. qPCR analysis showed that these genes are differentially expressed between pHGG and aHGG, with an average of 2.5-fold higher in SJ-GBM2 and CHLA-200 cells compared to LN-229 cells (FIG. 1B). The results showed that, miR-20a was most highly expressed miRNA among the miR-17-92 gene cluster members in the pGBM cell lines and that miR-20a was aberrantly expressed as in pGBM as compared to aGBM and normal astrocytes (FIG. 2A). This result was also observed in pediatric patients with high-grade glioma (pHGG) (FIG. 2B). Moreover, miR-20a expression in pGBM cells was the highest among other solid tumors tested (aGBM, breast, ovarian, pancreas, colon, prostate cancer, and melanoma cells, FIG. 2C). These results showed a distinct disparity in the aberrant upregulation of miR-20a in the pediatric population with GBM, which suggests that this molecule can be a specific and attractive therapeutic target for pGBM.

### Example 2: a Conjugated Complex Consisting of p28 and Antisense miR-20a (AmiR20-p28) Preferentially Penetrates Cancer Cells

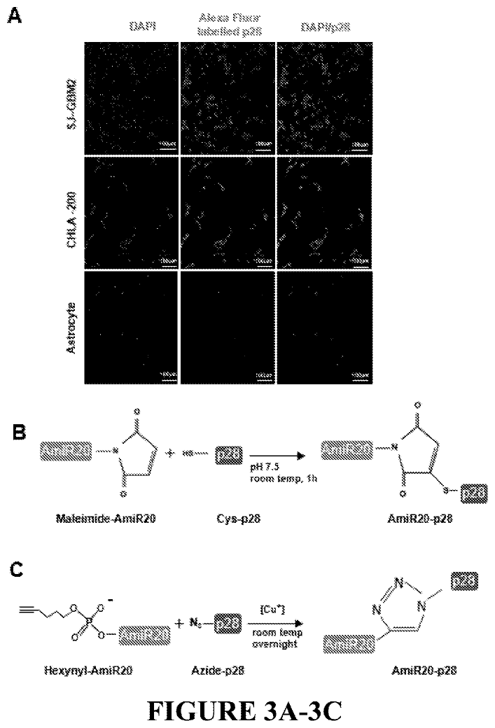

Our previous findings showed that a CPP, p28, preferentially enters various human cancer cells over noncancerous cells. As shown in FIG. 2D, p28 preferentially penetrated SJ-GBM2 and CHLA-200 cells compared to normal astrocytes (higher magnification images in FIG. 2D and lower magnification images in FIG. 3A). To utilize the ability of p28 to specifically target tumor cells, we created a new agent that combines the tumor-targeting capacity of p28, a CPP, with the anticancer activity of antisense miR-20a by covalently linking p28 and antisense miR-20a (AmiR20-p28) to efficiently deliver antisense miR-20a and target miR-20a in pGBM. Maleimide-antisense miR-20a and Cys-p28 were conjugated through a disulfide bond (FIG. 3B). First, the cellular entry of AmiR20-p28 was assessed with the same set of cell lines. Confocal images showed preferential entry of AmiR20-p28 in pGBM cells compared to normal human astrocytes (FIG. 2E). The intracellular entry of AmiR20-p28 was also quantitatively confirmed by the detection of antisense miR-20a. qPCR analyses indicated the preferential and dose-dependent penetration of AmiR20-p28 into pGBM and aGBM cells (FIG. 2F). The end products following qPCR analysis of SJ-GBM2 cells, CHLA-200 cells, LN-229 cells, and normal human astrocytes treated with AmiR20-p28 were subsequently visualized on agarose gels. This assay confirmed the cellular entry of AmiR20-p28 in pGBM and aGBM cells, whereas very little signal was detected in normal human astrocytes (FIG. 2G). These results show that p28, a tumor-targeting CPP, can efficiently deliver a cargo molecule, antisense miR-20a, in a dose-dependent manner.

### Example 3: AmiR20-p28 Downregulates Oncogenic miR-20a and Inhibits Cell Growth

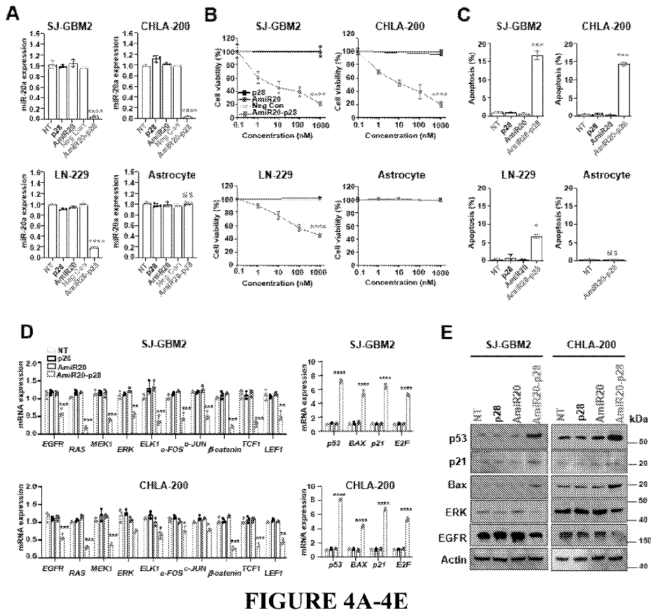

To determine whether AmiR20-p28 sequesters miR-20a and decreases endogenous miR-20a levels, we measured miR-20a expression levels in pGBM cells, aGBM cells, and normal human astrocytes. The qPCR results showed that miR-20a expression levels were significantly reduced by 94%, 96% and 84% in SJ-GBM2, CHLA-200 and LN-229 cells, respectively. The miR-20a levels in normal human astrocytes were not significantly altered, probably due to the characteristics of AmiR20-p28 preferential entry (FIG. 4A). In contrast, the two components of AmiR20-p28, the antisense miR-20a oligonucleotide (AmiR20) alone and p28, did not have a significant inhibitory effect in any of the cell line tested when administered alone (FIG. 4A). This result suggests that the significant miR-20a silencing in GBM was due to the effect of antisense miR-20a delivered by p28.

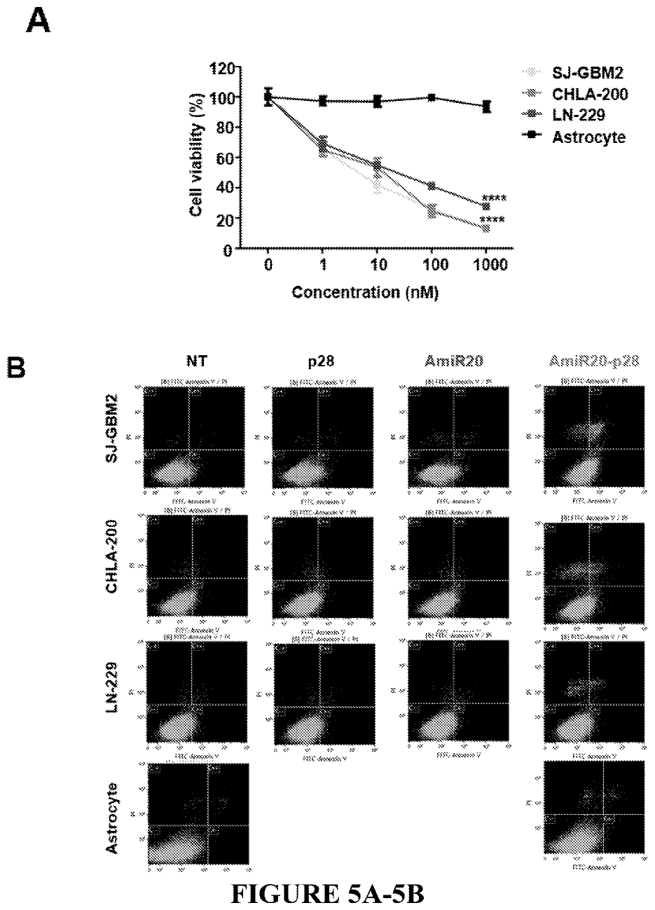

Next, we examined whether silencing miR-20a by AmiR20-p28 would promote any inhibitory effects on cancer cell viability in vitro. Cell Counting Kit-8 (CCK-8) viability assays showed that AmiR20-p28 significantly decreased the viability of pGBM cells (SJ-GBM2 and CHLA-200) (FIG. 4B). In contrast, the effect of AmiR20-p28 on cell viability was less extensive in aGBM (LN-229) cells (compared to pGBM) and negligible in normal human astrocytes (FIG. 4B). These results are correlated with the degree to which miR-20a was silenced in the cells. Again, neither p28 nor AmiR20, alone had an inhibitory effect on cell viability. This finding is consistent with the report that p28 alone demonstrates inhibitory effects on cancer cell viability at a higher dose than what was tested in this study (50 μM) in GBM cells. Notably, AmiR20-p28 at doses as low as 10 nM decreased cell viability to ~45% and 50% in SJ-GBM2 and CHLA-200 cells, respectively. The IC₅₀ values for AmiR20-p28 in SJ-GBM2, CHLA-200 and LN-229 cells were 8 nM, 11 nM, and 170 nM, respectively. To determine the inhibitory effects on cell viability was due to the chemical linker, we prepared AmiR20-p28 in a different chemical conjugation way by click chemistry (FIG. 3C). Given that similar inhibitory effects on cell viability in the same set of cell lines were obtained with AmiR20-p28 prepared by click chemistry in the set of cell lines tested (FIG. 5A), such effects were not due to the linker used for the conjugation process.

Furthermore, we performed flow cytometric analyses to detect apoptotic cells using Annexin V/propidium iodide (PI) staining. Among the pGBM cells exposed to 10 nM AmiR20-p28 for 24 h, SJ-GBM2 cells showed a significant increase in apoptotic cells by 16%, and the number of apoptotic cells was significantly increased by 14% in CHLA-200 cells, but not control groups (NT, p28 alone, AmiR20 alone) (FIG. 4Cand FIG. 5B). In contrast, 6.7% and 0.5% of aGBM and astrocytes, respectively, were apoptotic after AmiR20-p28 treatment. As shown in FIG. 2A-2C, oncogenic miR-20a is aberrantly expressed in GBM, particularly pGBM. As expected, AmiR20-p28 treatment had little effect on the viability of MDA-MB231, SKOV3, Mia-Paca2, Colon 205 and DU145 cells, suggesting that AmiR20-p28 specifically affects pGBM cells, in which miR-20a is aberrantly expressed (FIGS. 2A-2C and FIG. 5C). These results demonstrate that AmiR20-p28 preferentially entered GBM cells and silenced miR-20a, thereby inhibiting cancer cell viability.

### Example 4: AmiR20-p28 Targets the Oncogenic Signaling Pathways in pGBM

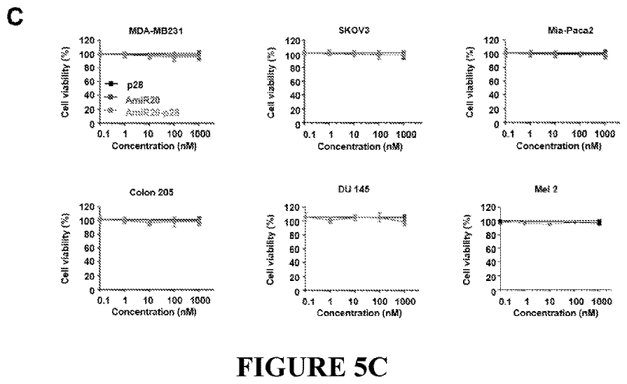

After demonstrating that AmiR20-p28 silences miR-20a and decreases cell viability in pGBM cells, we aimed to determine the mechanism through which this occurs. To elucidate the signaling pathways altered by AmiR20-p28, we determined the expression levels of downstream targets of miR-20a. Genes associated with the EGFR, p53 and TGF pathways were studied due to their critical roles in tumorigenesis signaling networks and miR-20a was suggested to regulate these genes. qPCR analyses showed that the expression levels of the EGFR, Ras, MEK1, ERK1, c-Fos, c-Jun, β-catenin, TCF-1 and Lef1 genes were significantly decreased after treatment with AmiR20-p28 but not with p28 alone, AmiR20 alone or PBS (NT), whereas a robust increase in the expressions of p53, p21, Bax, and E2F in SJ-GBM2 and CHLA-200 cells were observed (FIG. 4D). In LN-229 cells, AmiR20-p28 significantly increased the expression of p53, p21, Bax and E2F, and decreased EGFR expression levels, whereas no significant effect on gene expression levels was observed in normal human astrocytes (FIG. 6A and FIG. 6B). There is notable difference in the extent to which these genes are regulated between pGBM and aGBM (FIG. 4D and FIG. 6A). Genes in TGF and Wnt pathways such as β-catenin and TCF1 were significantly downregulated by AmiR20-p28 in pGBM, but not in aGBM LN229 (FIG. 4D and FIG. 6A). This difference between pGBM and aGBM may be due to significantly higher miR-20a expression levels in pGBM than aGBM, reflecting one decimal higher IC₅₀ in LN-229 cells than pGBM. To determine whether AmiR20-p28 affects expression of these genes at the protein level as well, we evaluated the protein levels of the major downstream targets of miR-20a. Consistent with the qPCR results (FIG. 4D), western blot analyses showed that the ERK and EGFR levels were decreased, whereas the p53, p21, and Bax levels were increased with AmiR20-p28 treatment compared to those in the controls (FIG. 4E). These results suggested that AmiR20-p28 can diminish oncogenic miR-20a expression and simultaneously alter multiple signaling pathways (FIG. 6C) by targeting and silencing ‘druggable’ and ‘undruggable’ genes, such as RAS, in pGBM.

### Example 5: AmiR20-p28 Exhibits Cytotoxic Activity Against Tumor Spheroids with Glioma Stem Cell Characteristics

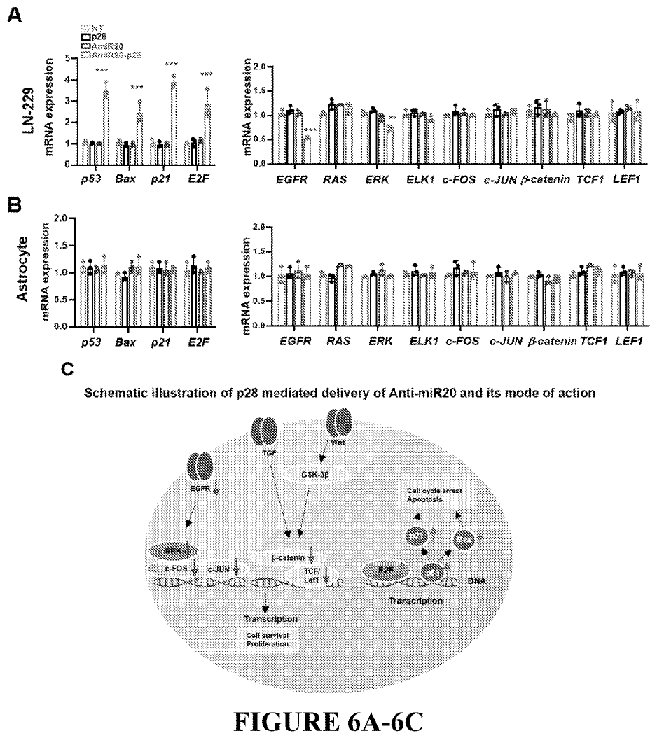

Pediatric brain tumors commonly recur due to the presence of tumor heterogeneity, which is often associated with drug resistance, and the self-renewal capacity of glioma stem cells (GSCs). To determine drug sensitivity, pGBM cells were cultured in the presence of temozolomide (TMZ), a chemotherapeutic agent for aGBM. A cell viability assay indicated that SJ-GBM2 cells had a significantly lower susceptibility to TMZ (IC₅₀=~ 0.6 mM) than CHLA-200 (FIG. 7A). In addition, as SJ-GBM2 cells positively stained for the most common surface marker for GSCs, CD133 (FIG. 7B).

In general, monolayer cell cultures are used to assess the potency of dissolved agents that are readily accessible in the cell medium. Native tumors, in contrast, contain densely packed cells surrounded by other cells. To better mimic the complex tumor structure, we generated tumor spheroids using SJ-GBM2 cells. To test the penetrative ability of AmiR20-p28, we first explored its uptake in the SJ-GBM2 tumor spheroids. Confocal images showed that AmiR20-p28 penetrated the tumor spheroids (FIG. 7C). Following confirmation that AmiR20-p28 penetrates the tumor spheroids, the potential for AmiR20-p28 to affect the complex structure of tumors was examined in tumor spheroids. The structure of the SJ-GBM2 spheroids exposed to AmiR20-p28 started to disintegrate at 24 h, and this disintegration increased at 48 h (FIG. 7D), suggesting that AmiR20-p28 can penetrate 3D culture and disrupt the GSC spheroid structure. These in vitro results suggest that AmiR20-p28 can overcome the GSC characteristics of pGBM tumors if applied as a therapy in pGBM.

### Example 6: AmiR20-p28 Inhibits Orthotopic Glioblastoma Tumor Growth

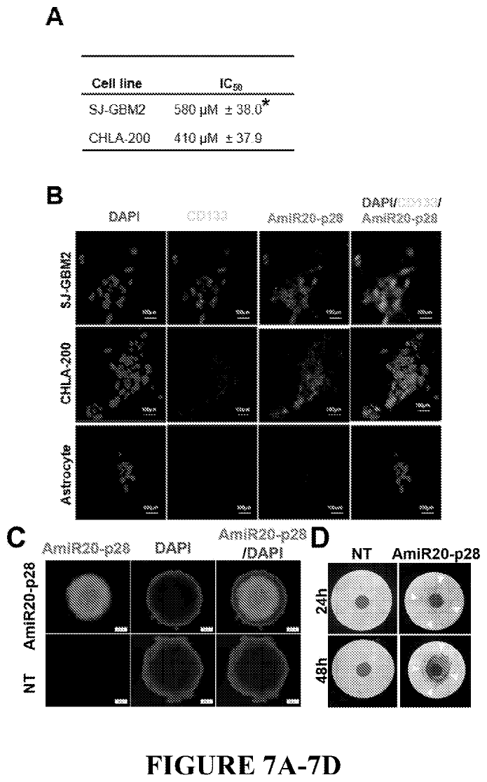

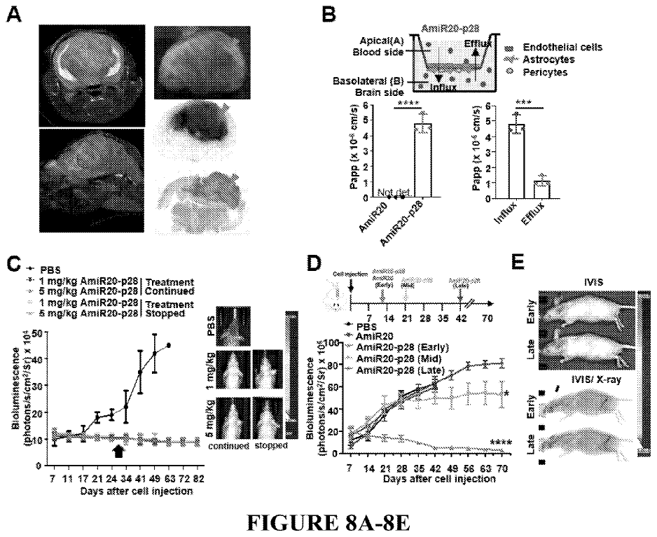

To determine the therapeutic efficacy and tumor inhibitory capacity of AmiR20-p28 in vivo, we generated an intracranial orthotopic xenograft mouse model by injecting SJ-GBM2 cells, and tumor development was tested. SJ-GBM2 tumors were conspicuous in T2-weighted magnetic resonance (MR) images, and histological examination also showed tumor-positive areas in the coronal section of the brain (FIG. 8A). After systemic intravenous (i.v.) administration of indocyanine green (ICG) dye conjugated with p28 in the mice, near-infrared (NIR) fluorescence images of the brain showed that p28 preferentially localized at the tumor lesions (FIG. 8A and FIG. 9). The ability of a vehicle to cross the BBB is the major challenge for gene delivery. Here, we tested the ability of AmiR20-p28 to cross the BBB using 3D human BBB assay kits in vitro. The BBB permeability of AmiR20-p28 and AmiR20 in two directions, apical to basolateral (A-B) and basolateral to apical (B-A) was measured. Quantitative analysis using qPCR showed that AmiR20-p28 crossed the BBB at a significant rate (FIG. 8B). In contrast, AmiR20 was undetected, suggesting that the ability of AmiR20-p28 to cross the BBB was due to the p28 motif. As high import (blood to brain) and low export (brain to blood) of AmiR20-p28 would be desirable for the drug to accumulate in the brain, we determined the AmiR20-p28 influx and efflux rates. The influx Papp (A-B) and efflux Papp (B-A) rates were 4.39×10⁻⁶ and 0.95×10⁻⁶ cm/s, respectively (FIG. 8B). The influx transport rate of p28 to the brain was higher than the efflux transport rate suggesting that AmiR20-p28 is an ideal BBB-permeable agent.

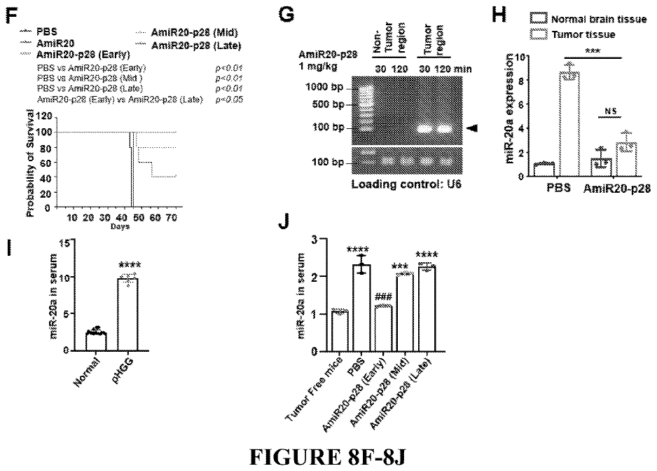

After determining that AmiR20-p28 can successfully cross the BBB in vitro, we aimed to investigate the therapeutic effect of AmiR20-p28 in vivo, SJ-GBM2 cells genetically modified to express luciferase (SJ-GBM2-luc cells) were implanted in mice and monitored for luciferase signaling via bioluminescence intensity imaging. Following systemic treatment with 0.1, 1 or 5 mg/kg AmiR20-p28 i.v., there was a significant inhibition of tumor growth compared to that of the mice treated with the negative control (PBS) or the lowest does (0.1 mg/kg) of AmiR20-p28 (FIG. 8C and FIG. 10). After four weeks of treatment consisting of 12 injections per mouse administered three times per week, the groups receiving AmiR20-p28 at 1 and 5 mg/kg were further divided into two subgroups: one group received continued treatment (3× weekly i.v), while the other group stopped treatment to see if tumors recurred. Throughout the additional ~two months experimental period, neither the 1 mg/kg nor the 5 mg/kg dose groups exhibited any signs of tumor recurrence (FIG. 8C).

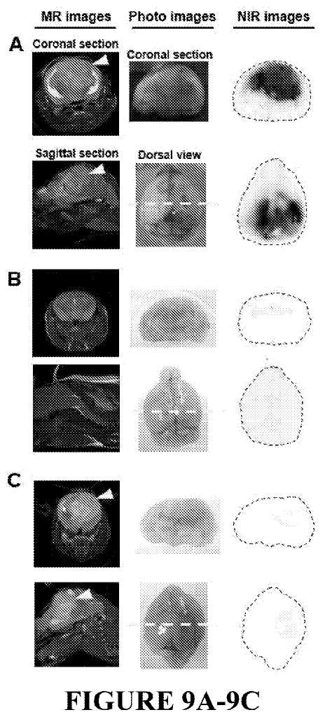

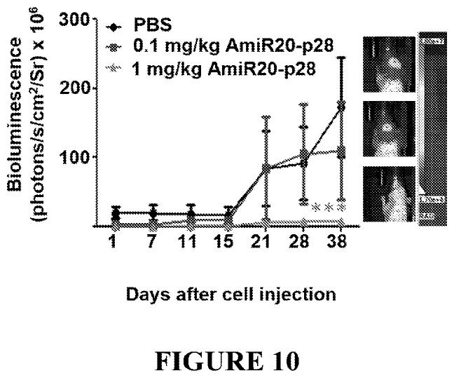

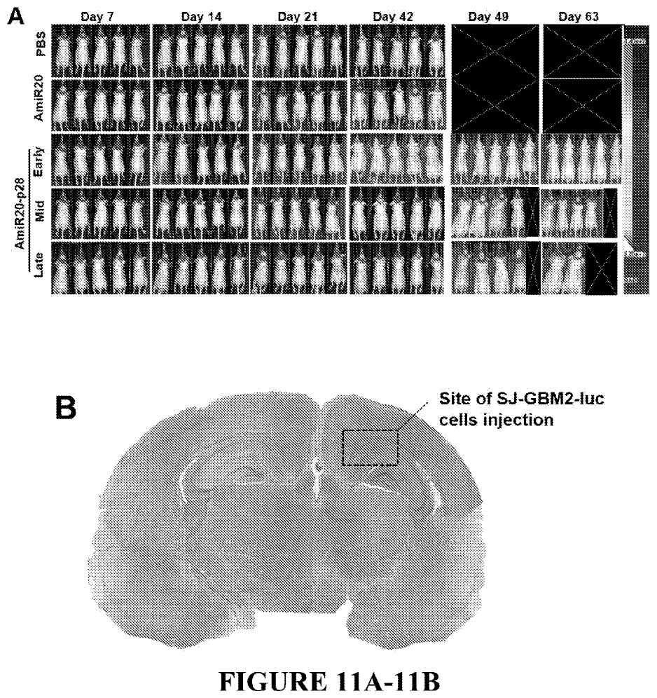

Subsequently, another set of experiment was performed to validate and evaluate the potency of AmiR20-p28 in regulating tumor growth at various stages/sizes. AmiR20-p28 i.v. treatment at a minimum effective dose (1 mg/kg) was initiated at three different time points at 10 (early phase), 20 (middle phase), and 40 (late phase) days after cell injection to the mice (FIG. 8D, upper panel). Consistent with the previous experimental results (FIG. 8C), the mice treated with AmiR20-p28 at the early phase of tumor development showed complete repression of tumor (FIG. 8D, FIG. 8E, FIG. 11A-11B). The mice treated with AmiR20-p28 at the middle phase of tumor development showed sustained suppression of the tumor burden (FIG. 8D, FIG. 11A). Although tumor progression was observed in the late-phase animals, 80% of mice in this group survived at day 49, unlike those in the control groups (PBS and AmiR20) (FIGS. 8D-8F, and FIG. 11A). Mice died due to disease or were sacrificed based on the humane endpoint criteria, including abnormal behavior (e.g., paralysis) (FIG. 12). Based on the results of toxicity tests [a hepatotoxicity assay, as miRNA is known to be metabolized in the liver (FIG. 13A-13B), a hemolysis assay, as AmiR20-p28 was injected via i.v. (FIG. 13C)], and histological examination of major organs (FIG. 13D), AmiR20-p28 induced antitumor effects without apparent adverse effects. The overall survival in the AmiR20-p28 treatment groups was associated with their anti-tumor effects; the overall survival rates were 100% (early phase), 80% (middle phase), and 40% (late phase), which differed from those of the control groups administered PBS or AmiR20 (FIG. 8F).

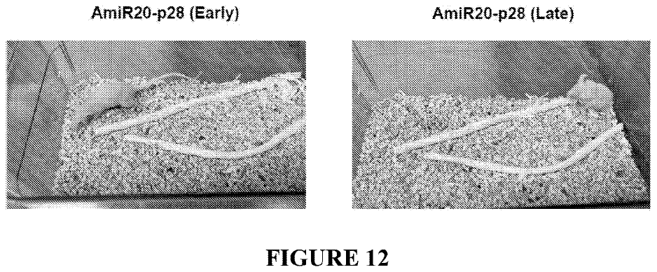

To evaluate whether systemically delivered AmiR20-p28 can localize at tumor lesions in in vivo, we measured AmiR20-p28 in normal brain tissue and tumor tissue. Mice bearing SJ-GBM2 tumors were injected with 1 mg/kg AmiR20-p28 for 30 or 120 min. Brain samples were isolated and separated into normal brain tissue and detectable tumor tissue by bioluminescence imaging and quantitative human-specific Alu sequencing (huAlu), which can identify human-derived SJ-GBM2 cell tumors implanted in mice, as Alu-repeat DNA sequences are specific to human cells (FIG. 14A-14B). The tumor and normal brain tissues were subjected to qPCR to detect AmiR20-p28. Even 30 min after the injection, substantially higher levels of AmiR20-p28 were detected in tumor tissues than in normal brain tissues (FIG. 8G). These results showed that AmiR20-p28 is the BBB-permeable agent with high influx/low efflux rates and effective targeted agent for pGBM in preclinical settings.

To determine whether AmiR20-p28 tumor localization has the biological effects on the miR-20a levels, mice were treated with PBS or 1 mg/kg AmiR20-p28 3 times a week for one week since long-term exposure to AmiR20-p28 resulted in tumor regression. Brain samples were isolated from the PBS- and AmiR20-p28-treated mice and separated into normal brain tissue and tumor tissue. A significant decrease in miR-20a levels was detected in the tumor tissues resected from the AmiR20-p28-treated mice compared to those from PBS-treated mice (FIG. 8H), which remained similar to those of the original SJ-GBM2 cells (FIG. 2C). Together, these results confirmed the in vivo efficacy and tumor specificity of AmiR20-p28 in inhibiting oncogenic miR-20a.

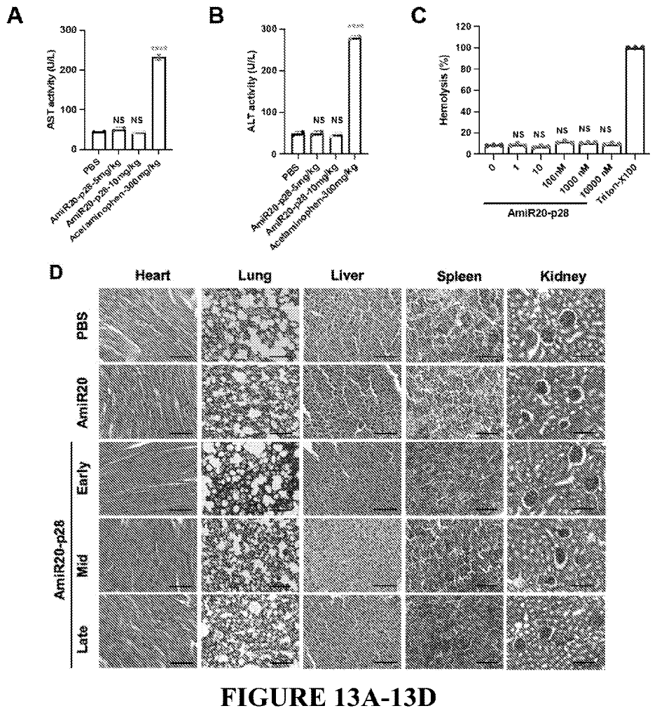

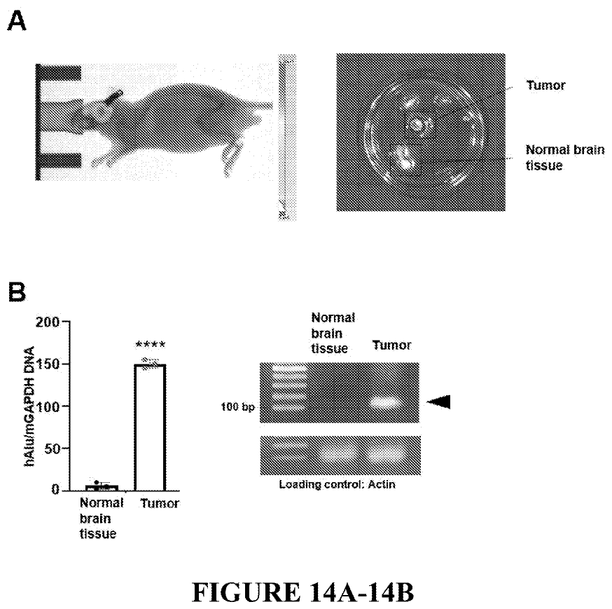

One recent emerging area for cancer diagnosis and prognosis is noninvasive, circulating serum biomarkers development. To investigate whether the tumor burden correlates with circulating oncogenic genes, we measured miR-20a in human sera. Evaluation of circulating miR-20a levels showed that they were significantly higher in patients with pHGG than in healthy volunteers (FIG. 8I). This result indicates that serum miR-20a could serve as a pGBM biomarker. To further examine serum miR-20a levels in the in vivo animal model described above (FIG. 8D), we analyzed miR-20a levels in serum samples from the PBS and AmiR20-p28 treatment groups (animals from the experiments presented in FIG. 8D) and compared them to those in serum samples from the tumor-free control mice. As expected, qPCR analyses demonstrated that the miR-20a level was significantly higher in the PBS treatment group than in the tumor-free group (FIG. 8J) which were consistent with the results in FIG. 8I. More importantly, the serum levels of miR20a were correlated with tumor burden (FIG. 8J, and FIG. 15). The serum miR-20a level in the early-phase group was not significantly different from the basal level in the tumor-free mice (P=0.78), that circulating miR-20a levels can be used as a biomarker to monitor the tumor response (FIG. 8J). The absence of an elevated serum miR-20a level indicated that the tumors in these mice had completely disappeared in the early-phase treatment group. Overall, systemically administered AmiR20-p28 crossed the BBB and preferentially accumulated in orthotopic xenograft tumors. Without causing apparent adverse effects, AmiR20-p28 treatment inhibited tumor growth, which was associated with overall survival, that can be monitored by circulating miR-20a levels as a serum biomarker.

### Example 7: AmiR20-p28 Suppresses Tumor Growth in Orthotopic Glioblastoma

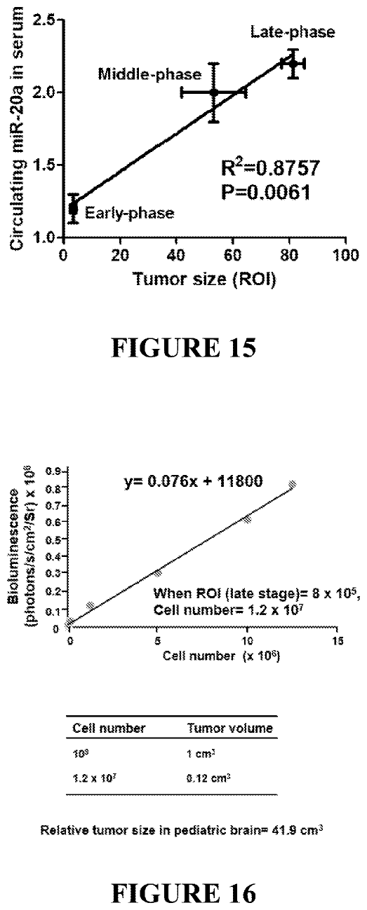

To determine the therapeutic efficacy and inhibitory capacity of AmiR20-p28 in vivo, an intracranial orthotopic xenograft mouse model was utilized for systemic delivery of anti-miR20. SJ-GBM2-luc cells, genetically modified to express luciferase were implanted and monitored for luciferase signal via bioluminescence intensity imaging in the control group and AmiR20-p28 treated mice. Following systemic treatment with AmiR20-p28 (1 mg/kg), there was a significant repression in tumor growth, as compared to the mice treated with negative control PBS and 0.1 mg/kg AmiR20-p28 (FIG. 17A). After AmiR20-p28 injections i.v., the luciferase activity from AmiR20-p28 treated mice was significantly lower than that of PBS control group, indicating that repeated treatment with AmiR20-p28 reduced the rate of glioblastoma growth. To evaluate the potency of AmiR20-p28 in controlling the size of tumor growth, mice were treated with 1 mg/kg AmiR20-p28 i.v. at three different time points at 10 (early), 21 (middle) and 42 (late phase) days after cell injection to the mice. As observed from previous experiment, mice treated with AmiR20-p28 at the early phase of tumor development at 10 days after cell injection showed complete repression of tumor (FIG. 17B). Moreover, AmiR20-p28 injected to mice which has bigger tumor mass developed after 21 days of cell injection showed sustained suppression of tumor growth (FIG. 17B). Mice with the late phase (42 days after the cell injection) of AmiR20-p28 treatment showed tumor progression (FIG. 17B). These anti-tumor effects of AmiR20-p28 were associated with their overall survival. AmiR20-p28 treatment resulted in increased overall survival rates with 100% (early phase), 80% (mid phase), and 60% (late phase) survival probabilities as compared to the control groups (PBS and Amir20 alone) (FIG. 17C). Furthermore, we detected the AmiR20-p28 uptake in the xenografted tumor tissues. Mice bearing SJGBM2 tumor were injected with 1 mg/kg AmiR20-p28 for 30 or 120 min. After the treatment, tumor regions and normal brain tissue regions were subjected to real time PCR. The results showed higher levels of AmiR20 were detected in the tumor regions (FIG. 17D). To further confirm AmiR20-p28 uptake in tumors, AmiR20-p28 labelled with Alexa fluor 568 was injected to mice bearing SJGBM2. The confocal images of the brain sections showed significant contrast of Alexa fluor signal in the tumor sites compared to normal brain regions (FIG. 17E). These results suggest that systemic administration of AmiR20-p28 preferentially accumulates at the given tumor in the mouse brain and AmiR20-p28 treatment enables to control tumor growth associated with increasing overall survival.

### Example 8: Effect of AmiR21-p28 on Various Solid Tumors

A cell-penetrating peptide, p28, was chemically conjugated to antisense-miR21 (such as AmiR21-p28). miR21 is known to be an oncogenic miRNA and highly expressed in various types of cancer cells.

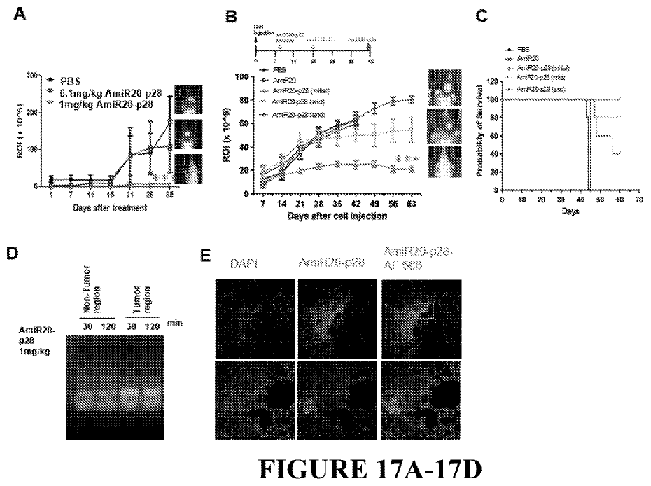

In contrast to miR20, adult glioblastomas (aGBM) LN229 showed the highest levels of miR21 compared to pGBM (FIG. 18A). Ten nM AmiR21-p28 exposure to pGBM and aGBM significantly reduced the endogenous miR21 (FIG. 18B-18D), but not in human normal astrocytes (FIG. 18E). p28 alone and antisense-miR21 (anti-miR21) alone did not induce a significant reduction of miR21 in any cell lines tested.

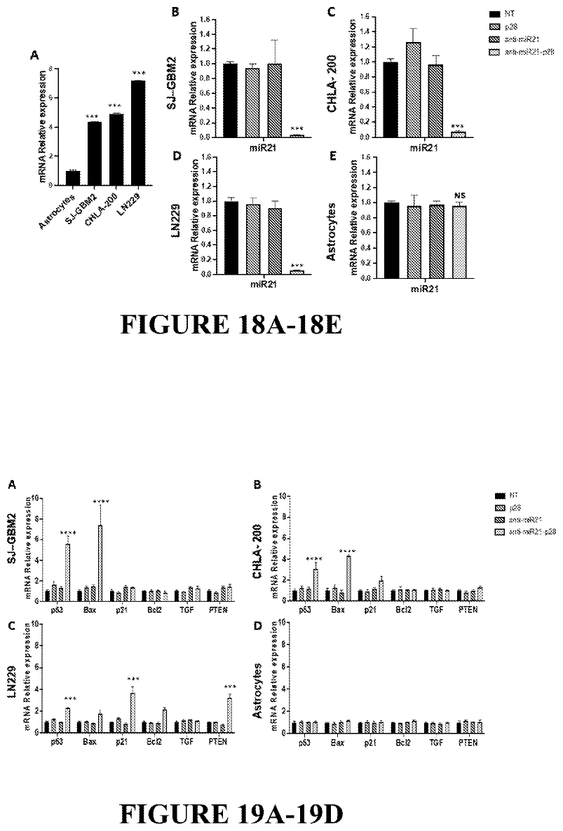

Alternations of gene expression profiles induced by AmiR21-p28 are different from those induced by AmiR20-p28 (e.g., PTEN), suggesting that target genes of miR21 is different from miR20 (FIG. 19).

We developed a novel miRNA delivery system using a cell-penetrating peptide p28 that can deliver agent across the BBB. AmiR20-p28 showed preferential penetration and exhibited cytotoxicity in pGBM cells. Importantly, we found that AmiR20-p28 simultaneously targets multiple signaling pathways to inhibit pGBM growth and improve the overall survival through the silencing of oncogenic miR20.

Despite the extensive studies in understanding the molecular profile of gliomagenesis in pediatric patients, the current knowledge is still insufficient to identify targets for developing therapeutic agents. In recent years, large number of evidence have shown that miRNAs play an important role in molecular and cellular mechanisms in tumorigenesis of various cancer including pGBM, that act either as oncogenes or tumor suppressors. Furthermore, miRNAs can simultaneously alter expression of multiple target genes and often disrupt entire signaling network. Gene expression profiling studies showed that the most elevated miRNAs in pediatric samples compared to adult population and normal brain tissues included: miR15a, miR17, miR18a, miR19a, miR19b, miR20, miR27a, miR-100, miR-106a, miR-195 and miR497. Increasing evidence has indicated that miR20 plays an important role as an oncogene in the development of a wide range of malignant tumors. In addition to, miR20 play a key role in glioma stem cells (GSC) invasion. These findings, in addition to their small size and influence in a broad range of biologic processes, makes miRNA an attractive therapeutic agent for pGBM. Another important factor in gene regulation is a small interfering RNA (siRNAs) which are investigated recently as novel classes of therapeutic agents for the treatment of a wide range of disorders including cancers. Clinical trials of siRNA and miRNA-based drugs have already been initiated. While both siRNAs and miRNAs have similarity consisting short duplex RNA exerting gene silencing, siRNA is highly specific for one mRNA target whereas miRNA can simultaneously alter expression of multiple target genes and often disrupt entire signaling networks, resulting in efficient changes in the activity of target cells. Therefore, our study approached by targeting miR20 for pGBM. However, the broad functionality of miRNA also could cause difficulty in controlling the off-target effects and toxicities. In addition, clinical application of miRNA as therapeutic agents is hindered by the limitation of delivery vector such as poor stability, low transfection efficiency, carrier associated toxicity generated from off-site target. Another huge obstacle in developing new drugs for CNS disorder is the delivery of the therapeutic agents across the BBB. The BBB represents a complex structure in the brain that controls molecular traffic, maintains homeostasis and keeps out toxins by selective transportation of nutrients and other molecules across the brain by gap junctions. Among the recent advances in the development of novel delivery system, transport of therapeutic molecules into the CNS through CPP-based delivery systems look more promising due to their great ability to carry macromolecules across cellular membranes with low cellular toxicity and high efficiency (Silva S et al., 2019). In this study, we use p28 delivery system which have shown to cross BBB in vitro and its application in human clinical trials which provides indirect evidence of p28 crossing BBB. Based on this previous finding, we conjugated anti-miR20 with p28 to inhibit miR20.

Conjugated complex of AmiR20-p28 showed preferential penetration in pGBM cell lines and suppressed miR20. This suppression of miR20 was associated with marked decrease in cell viability of pGBM cell lines than astrocytes cells. One of the important characteristic features of glioma is the disorder of the mechanism of natural programmed cell death. These observations indicate that AmiR20-p28 is tumor cell specific. p28 itself demonstrates significant cytotoxicity toward human carcinomas such as glioblastoma, melanoma, breast, prostate, colorectal etc. However, this effect of p28 in AmiR20-p28 treatment could be ruled out as shown by the absence of cytotoxicity in glioblastoma cells when treated with comparably smaller dose of p28 alone at 10 nM. In addition, the clinical findings where two phase I clinical trials of p28 (NCS745104) as a single agent in patients with advanced solid tumors and in pediatric patients reported to have no adverse effects, toxicity or immunogenicity emphasizes the safety of p28 as a carrier agent. It is now known that glioma stem cell (GSC) is one of the reasons for high level heterogeneity in a pediatric tumor associated with high recurrence rate. GSCs can both self-renew and differentiate as a means to repopulate tumors by producing more GSCs. GSCs in brain tumors have been shown to resistant to aggressive radiotherapy and largely unaffected by standard chemotherapies. Due to these aspects of GSCs, there exists a desperate need to find therapeutics that target these cells to reduce recurrence. In HGG stem cell population, CD133 is considered as a cell surface marker of stemness and has been widely used for identifying putative stem cells. SJ-GBM2 showed increased CD1333 expression level than astrocytes which suggest the spheroid formation ability shown by SJ-GBM2. Our data reveals that AmiR20-p28 is capable of penetrating and targeting the spheroids.

To understand the mechanism of AmiR20-p28, expression analysis of pGBM cells treated with AmiR20-p28 was determined. Together with the p53 axis, AmiR20-p28 regulated expression level of E2F. Dysregulation of EGFR is well established in adult glioblastoma which confers enhanced tumorigenicity and shows resistance to both radiotherapy and chemotherapy, it has been considered less important in pGBM. In this study, we observed significant downregulation of the EGFR pathway at multiple downstream signaling cascades, EGFR, Ras, MEK, ERK, ELK1 and c-fos. Despite the lack of universal expression in all pediatric brain tumors, EGFR overexpression is indeed observed more frequently in pGBM with datasets differing from 10% to 80%.

In the SJ-GBM2 pGBM orthotopic mouse model, complete regression was observed with early-phase treatment with AmiR20-p28. The relative tumor volume in the human pediatric brain was estimated to be ~40 cm³ based on mouse brain tumors at the early-phase, which suggests that AmiR20-p28 can theoretically be effective in pediatric patients with a golf ball-sized tumor (~4 cm). Hence, AmiR20-p28 treatment can feasibly be effective in early-phase pGBM patients and in even patients with relatively larger tumors after surgery as adjuvant therapy.

Additionally, the identification and development of new biomarkers to improve early cancer detection and monitoring drug efficacy are clinically important. For biomarkers, liquid biopsy is important due to its lower invasiveness and safer approach compared to traditional biopsies for diagnosis. Based on our preclinical evaluations, the level of miR-20a was significantly high in sera from pHGG patients and similar relevance was found in in vivo mouse experiments.

In summary, we highlight a new approach for miRNA-based therapy for pGBM utilizing p28 as a delivery platform. p28 facilitates the penetration of AmiR20 through the BBB and enables preferential intracellular delivery, resulting in endogenous miR20 suppression, regulation of multiple target proteins, tumor growth inhibition, and prolong overall survival. Additional systemic or neurological toxicity, pharmacokinetics, and biodistribution studies will determine the possibility of off-target or initiate designing early phase clinical trial. Overall, the results support p28 mediated delivery for miRNA-based approach for cancer treatment.

### Methods

### Cell Lines and Cell Culture

SJ-GBM2 (Female, 50 months, progressive disease, post-chemotherapy, histone 3 variant H3.3: wild type, TP53: R273C) and CHLA-200 cells (Male, 144 months, multiple recurrences treated with chemotherapy and radiation, histone 3 variant H3.3: wild type, TP53: wild type) were obtained from the Children's Oncology Group Cell Culture Repository (Lubbock, TX). Normal human astrocyte (NHA, CC-2565) was obtained from Lonza (Bend, OR). Human cancer cells (LN-229, MDA-MB231, SKOV3, MIA PaCa-2, Colon 205, and DU145 cells) were purchased from the American Type Culture Collection (Manassas, VA). SJ-GBM2 and CHLA-200 cells were maintained in IMDM supplemented with 20% fetal bovine serum (FBS), 100 units/ml penicillin, 100 μg/ml streptomycin, and 1% ITS. LN-229, MDA-MB231, SKOV3, MIA PaCa-2, Colon 205 and DU145 cells were maintained in MEME supplemented with 10% FBS, 100 units/ml penicillin, and 100 μg/ml streptomycin. Astrocytes were maintained in astrocyte growth media bullet kits (CC-3186). All cells were cultured at 37° C. in a humidified chamber containing 5% CO₂. Human melanoma (Mel-2) was developed in our laboratory as described.

### Preparation of AmiR20-p28

miRNA-peptide conjugation was conducted based on thiol-maleimide reactions. Antisense-miR-20a modified with a maleimide group at its 5′ terminal end was acquired from Integrated DNA Technologies (Coralville, IA). All RNA bases had 2′ O-methyl-modification. The sequence is 5′-maleimide CUA CCU GCA CUA UAA GCA CUU UA-3′ (SEQ ID NO:3). Cys-p28 (CLSTA ADMQG VVTDG MASGL DKDYL KPDD (SEQ ID NO:4)) was p28 with the addition of one cysteine group at the N-terminal end (CS Bio, CA). Maleimide conjugate antisense miR-20a was first deprotected by a retro Diels-Alder reaction by suspending 80 nmole of the lyophilized oligonucleotide in 2 ml of anhydrous toluene at 90° C. for four hours in absolute dry conditions and subsequent evaporation to obtain a white residue consisting of an active maleimide moiety conjugated with antisense miRNA. This ready form of the oligonucleotide was then reacted with 5 equivalents of thiol, 400 nmole, containing Cys-p28 in phosphate-buffered saline (PBS), and the final pH was maintained at 6.5-7.5 at room temperature for 1 hr. The conjugation reaction was subsequently confirmed with mass spectrometry (Bruker MALDI TOF Microfelx, Germany). A widely used negative control miRNA (5′-maleimide CAGUACUUUUGUGUAGUACAA-3′ (SEQ ID NO:5)) was obtained from Integrated DNA Technologies and conjugated with p28 as described above.

AmiR20-p28 was prepared with click chemistry conjugation between hexynyl-antisense miR20a and azide-p28. Hexynyl-antisense miR20a (5′-Hexynyl-CUACCUGCACUAUAAGCACUUUA (SEQ ID NO:6)) was purchased from Integrated DNA Technologies. Azide-p28 was obtained from Biomatik. 200 μl of 1 mM hexynyl-antisense miR20a in 500 μl D.W. It was prepared in a pressure tight vial such that its final concentration is 200 μM after adding solution in later steps. 100 μl 2M triethylammonium acetate buffer (TEA) pH 7.0 was added to the solution followed by addition of 20 μl DMSO. 30 μl of 10 mM azide-p28 was added to the solution and vortexed. Then, 100 μl of 5 mM ascorbic acid solution (AlfaAesar, #A15613.18) was added to the mixture which was degassed by bubbling argon for 30 sec. Mixture was then followed by addition of 0.5 mM Cu-TBTA complex (Lumiprobe, #21050) and degassed. Reactions were incubated at room temperature with gentle agitation overnight. The conjugate was precipitated with acetone, at least 4-fold volume of the mixture, and kept at −20° C. for 20 min and centrifuged at 10,000 r.p.m. for 10 min at 4° C. Pellet was washed again with acetone and dried at room temperature. Finally, the pellet was reconstituted in PBS and stored at −80° C. The conjugation reaction is subsequently confirmed with mass spectrometry.

### Quantitative Real Time PCR Assay

Micro RNA enriched fraction was extracted using the miRNeasy Mini Kit (Qiagen, Catalog #217004) following the manufacturer's instructions. Stem loop real-time qPCR was used to analyze miRNA expression. We designed miR-20a stem loop RT primers and amplification primers according to a previously described method 79. cDNA was synthesized from extracted RNA using unique stem loop RT primers. The sequence of the stem loop primer for miR-20a was 5′-GTCGTATCCAGTGCAGGGTCCGAGGTCGGCAATTGCACTGGATACGACCTACCT-3′ (SEQ ID NO:7). Reverse transcriptase reactions contained the following reagents: 10 ng of RNA sample, 60 nM stem-loop RT primer, 1×RT buffer, 0.25 mM dNTP, and 4 U/μl reverse transcriptase. Reactions were performed by incubation at 16° C. for 30 min, 42° C. for 30 min and 85° C. for 5 min. Real-time PCR (7300 Real time PCR Systems, Applied Biosystems, CA) was performed using the specific forward primer for miR-20a (5′-GTAAAGTGCTTATAGTGCAG-3′ (SEQ ID NO:8)) and a universal reverse primer (5′-GTGCA GGGTC CGAGGT-3′ (SEQ ID NO:9)). The 20-μl PCR mixture included 1×SYBR Premix (Invitrogen), 2 μl of RT products and each forward and reverse primer at 10 nM. Reactions were performed by incubation in a 96-well plate at 95° C. for 30 sec, followed by 35 cycles of 95° C. for 30 sec and 60° C. for 30 sec. U6 was used as an internal control to normalize the level of target miRNAs. The results were verified by performing PCR using M13 modified forward and reverse primers specific to stem loop sequence. The PCR products were visualized in 2% agarose gels and DNA-sequence was confirmed. Human tumor and serum samples were obtained from patients who had signed informed consent and the institutional review board approval was obtained in Pediatric Brain Tumor Consortium (PBTC-041) participating sites.

p28/AmiR20-p28 Labeling

p28 or AmiR20-p28 were dissolved in PBS mixed with Alexa Fluor 568 dye (Invitrogen) at a 1:2 peptide/dye ratio 37. Sodium bicarbonate (pH 8.5) was added and incubated at 4° C. with continuous stirring. Alexa Fluor 568 labeled p28 and AmiR20-p28 were dialyzed against cold-PBS using Slide-A-Lyzer Dialysis Cassettes (Pierce Biotechnology). Similarly, p28 was labeled with ICG as described previously.

### In Vitro BBB Assay

Permeability experiments were carried out in a 3D Human Blood Brain Barrier Model kit (Alphabioregen, Catalog #EP010). The BBB kits were activated following the manufacturer's instructions. Briefly, the BBB model was thawed using BBB growth medium (#BBB-GM001) and incubated overnight with fresh BBB growth medium. The model was incubated with Endo-Neuro-Pharmaceuticals medium (#NMBBB001) for 3 days. AmiR20-p28 was prepared in assay buffer (Dulbecco's PBS containing CaCl2 and MgCl4, 100 mM HEPES, pH 7.0, 25 mM D-glucose). The permeability of AmiR20-p28 was measured in two directions: apical to basolateral (A-B) and basolateral to apical (B-A). The apical side represented the blood, and the basolateral side represented the brain tissue. For transport, the inserts from the BBB wells were transferred to another well containing prewarmed assay buffer. Depending on the direction, the medium in the donor compartment of the BBB model was replaced with AmiR20-p28. The plate was incubated for 30 min at 37° C. Samples were then collected from the donor and acceptor compartments and analyzed by real-time PCR. The concentrations of AmiR20-p28 in the compartments were calculated based on a standard curve developed with AmiR20-p28 at 1, 10, 100 and 1,000 nM and its relative Ct value obtained from real-time PCR. The apparent permeability (Papp) coefficients in cm/s were calculated according to the following formula: Papp=(dC/dT×V)/(A×C₀) where dC/dt is the change in concentration in the basal chamber (μg/sec), V is the volume of the basal chamber, A is the membrane surface area and C₀ is the initial concentration in the apical chamber at 0 min. The efflux ratio (ER), defined as the ratio of Papp (B-A)/Papp (A-B), was used to estimate the magnitude of efflux.

### Cell Viability Assay

Cell viability was evaluated by a CCK-8 assay (Dojindo, Japan). Cells were seeded at a density of 4,000 cells per well in 100 μl of growth medium in 96-well plates and grown overnight. Then, the cells were exposed to antisense miR-20a alone, p28 alone or negative control or AmiR20-p28 over a range of concentrations and incubated for 24 hr. According to the manufacturer's instructions, 10 μl of CCK-8 solution was added to each well, followed by incubation for 1 hr at 37° C. The absorbance at 450 nm was determined by a multiplate reader.

### Cell Viability was Calculated as Follows:

Cell viability (%)=Abs(sample)−Abs(blank)/Abs(no treatment)−Abs(blank)×100

### Apoptosis Assay

Cells were seeded into 10-cm dishes and cultured overnight. Then, the cells were treated with 10 nM antisense miR-20a alone, p28 alone or AmiR20-p28 for 24 hr. Cells were washed with 1×PBS, detached with TrypleE (Invitrogen). and collected by centrifugation. Cells were washed and resuspended with 1× Annexin binding buffer (Life Technologies, catalog #V13242), and the cell density was maintained at 10⁶ cells/ml. Subsequently, 5 μl of Annexin-V was added to 100 μl of the cell suspension, and the mixture was incubated for 15 min at room temperature. The cells were washed and incubated with 5 μl of PI for an additional 15 min on ice, washed and resuspended in 200 μl of buffer. Cells without treatment were used as controls. After washing, cells were subjected to flow cytometry (Gallios flow cytometer) and analyzed with Kaluza software to give the proportions of dead (top left quadrant), late apoptotic (top right quadrant), early apoptotic (bottom right quadrant) and viable (bottom left quadrant) cells.

### Western Blot Analyses

Whole-cell lysates were prepared with RIPA buffer (Cell Signaling Technology, #9806S) according to the manufacturer's protocol. The total protein was quantified with Bradford reagent (Bio-Rad), and the proteins were subjected to one-dimensional gel electrophoresis with the NuPAGE system and then transferred to nitrocellulose membranes (Bio-Rad). Nitrocellulose membranes were incubated in blocking buffer (Thermo Scientific, #37535) for 2 hr at room temperature and then blotted with each primary antibody (anti-p53 at 1:1000, Santa Cruz SC-17846; anti-p21 at 1:1000, Santa Cruz SC-397; anti-ERK at 1:1000, Santa Cruz SC-94; and anti-Bax at 1:1000, Santa Cruz SC-20067) and anti-EGFR (1:1000; Invitrogen MA5-13070) at 4° C. overnight. The membranes were washed and incubated with HRP-conjugated secondary antibodies. Each band was visualized using Pierce SuperSignal West Pico Chemiluminescent substrate (Thermo Fisher Scientific). Anti-actin (1:1000, Santa Cruz SC-1616) was used as an internal loading control.

### Intracranial Tumor Implantation

All animal experiments were performed in accordance with the use of animals in research and approved by IACUC. SJ-GBM2 cells stably expressing luciferase gene (pGL4.51 [luc2/CMV/Neo], Promega) were generated by a chemical transfection method (FuGENE HD, Promega) in the presence of G418 antibiotics (GoldBio). 4-5 weeks athymic mice were purchased from The Jackson Laboratory. SJ-GBM2-luc cell lines was maintained to 70% confluency and checked for their luminescence activity. Similar to the implantation method described previously, cells were resuspended in PBS at a final concentration of 10⁶ cells/3 μl. Following anesthesia, a midline incision was made on the calvarium, extending from bregma to lambda sutures. At a position 2 mm posterior to bregma and 2 mm lateral to the coronal suture, a 3-mm hole was made in the skull. A 26-gauge Hamilton syringe was inserted into the hole, and 3 μl of 10⁶ cells was injected slowly. The injection site was covered with sterile bone wax and sutured. As the first set of experiment, mice were randomly divided into the experimental groups and the dose finding studies were conducted with AmiR20-p28 at 0.1, and 1 mg/kg. AmiR20-p28 was administered every other day three times a week i.v. via the tail vein (200 μl/injection). As the second set of experiment, mice were randomly divided into the groups of PBS or AmiR20-p28 at 1 and 5 mg/kg (N=5). After four weeks of treatment (total 12 injections/mouse, three times per week, i.v), the AmiR20-p28 groups at 1 and 5 mg/kg were further divided into two subgroups: one group received continued treatment (3× weekly i.v), while the other group did not receive any treatment to see if tumors recurred over the additional ~two months experimental period. As the third set of experiment, 1 mg/kg AmiR20-p28 was administered three times a week i.v. at three different tumor growth phases [at 10 (early phase), 20 (middle phase), and 40 (late phase) days after cell injection]. Mice were sacrificed based on the following humane endpoints such as >20% weight loss, the presence of labored respiration, abnormal behavior (e.g., paralysis), and loss of the ability to ambulate.

### In Vivo Bioluminescence Imaging

D-Luciferin potassium salt (GoldBio, #LUCK-100) at 150 mg/kg was injected intraperitoneally into the mice. Mice were anesthetized in a chamber with isoflurane/oxygen and positioned for bioluminescent imaging using a Spectral Lago X imaging system (Accela, Czech Republic). A series of images was acquired over a duration up to 30 min after the time of D-luciferin injection. Aura software (version 3.2) was used to compute regions of interest (ROIs) and integrate the total bioluminescence signal in each ROI. Data were analyzed based on radiance (photons/second/cm²/steradian) in the ROIs. The radiance values were plotted for each animal in the experimental group to form the tumor growth curve. Overall survival was also evaluated based on their survival period (mice died due to disease or were sacrificed based on the humane endpoints criteria). For X-ray imaging, the anesthetized mice were positioned, and a Spectral Lago X imaging system was used to take images. Aura software was used to merge the images taken with X-ray and bioluminescent systems.

### Confocal Microscopy

SJ-GBM2 cells, CHLA-200 cells and astrocytes (2×10⁴ cells/well) were cultured on glass slides placed in 24-well plates. After overnight culture, the cells were washed with PBS and incubated with 20 μM Alexa Fluor 568-labeled p28 peptide in medium (5% FBS and without phenol red) for 2 hr at 37° C. Cells were fixed with 2% formalin for 10 min. After washing, slides were prepared and mounted with Vectashield with DAPI (Vector Laboratories, Catalog #94010). Slides were examined by an LSM 710 confocal laser scanning microscope (Zeiss). Images were processed using ZEN software (ZEISS ZEN Lite). As for a GSC marker, the slides were first incubated with Alexa Fluor 568-labeled AmiR20-p28 for 2 hr at 37° C. Cells were fixed with 2% formalin for 10 min. After washing, slides were incubated with blocking buffer for 1 hr at room temperature, followed by consecutive staining with anti-CD133 antibody (1:500, R&D Systems FAB11331G). Slides were washed and mounted with Vectashield containing DAPI. Slides were examined by the LSM 710 confocal laser scanning microscope.

### Histological Analyses

Collected samples were fixed with buffered 3.7% formalin (Anatech) for 24 hr. Formalin was replaced with 70% ethanol following fixation. Samples were paraffin-embedded, and blocks were cut into 4 μm-thick sections and mounted onto slides which were processed for H&E staining. H&E-stained slides were analyzed by a pathologist blinded to the experimental groups.

### Blood Collection

Whole blood samples (~500 μl) were collected by cardiac puncture into tubes before sacrifice. To perform cardiac puncture, mice were deeply anesthetized under isoflurane and a 21-gauge needle was inserted into the heart. Mice were euthanized immediately following the cardiac puncture. After collection of whole blood, it was allowed to clot by leaving it undisturbed at room temperature for 15 minutes. The clot was then removed by centrifuging at 2,000×g for 10 minutes at 4° C. Human serum samples from healthy volunteers were obtained from Discovery Life Sciences (Huntsville, AL). Sera from pediatric patients with GBM (ID #16) and malignant glioma (ID #17) were from our previous study. Circulating miRNA in sera was extracted by NucleoSpin miRNA extraction kit (Takara, #740971) following the manufacturer's instructions. Stem loop real-time qPCR was used to analyze miRNA expression.

### Statistical Analysis

GraphPad Prism 9.0 was used for statistical analysis of data. Two tailed Student t tests were used for single comparisons, and group differences were evaluated using ANOVA. The survival assay was analyzed using Kaplan-Meier Test. Data were presented as mean±SEM, and all experiments were performed in triplicates unless otherwise mentioned.

Differential miRNA Expression Analyses

The dataset analyzed in this study is available in the GSE42657 repository. Data was processed with a significant cutoff at the p value<0.05. The miRNA expression dataset includes five grade IV pGBM (age range 4-15 years old) and two control tissues (age range 21-22 years old).

### MR Imaging

MR images of the brain were recorded by a 9.4 T MRI system (Agilent, Santa Clara, CA) as described before 80. Briefly, mice were anesthetized with isoflurane/oxygen, temperature was maintained and respiration was monitored throughout the entire scan. T2-weighted MR images were acquired using a fast spin-echo sequence with the following acquisition parameters: TR/TE 2050/8 ms, echo train length 8, matrix 128×128, FOV 19.2 mm×19.2 mm, slice thickness 1 mm. Images were visualized by using MirocDicom (ver. 2022.1).

### Tumor Spheroid Formation

SJ-GBM2 spheroids were formed by self-aggregation of cells in the bottoms of non-adherent round bottom 96-well plates (Thermo Scientific, #174925 96U). Suspensions of 10,000 cells in 100 μl volumes of media were pipetted into individual wells and allowed to form cell-cell connections over the course of 3 days.

### NIR Fluorescence Imaging

Mice bearing tumors in the brain were injected with ICG labeled p28 at 0.5 mg/kg. After 24 hr, the brains were scanned by the Odyssey imaging system (Li-cor, NE). Specific NIR signals at 800 nm were recorded.

### Toxicity Assays

Hemolysis assay: Potential hemolytic activity of AmiR20-p28 was determined as before. Briefly, human whole blood samples were centrifuged for 10 min at 1,000×g, the pellets washed with PBS and HKR buffer (pH 7.4), resuspended in HKR buffer, and 10 μl transferred to tubes with 190 μl of AmiR20-p28. Triton X-100 detergent at 0.1% was used as a control to disrupt the RBC membrane. After 30 min, tubes were centrifuged and absorbance (540 nm) of supernatants were recorded. Hemoglobin release in the presence of Triton X-100 was defined as 100%.

### Analyses of Liver Enzymes

Alanine transaminase (ALT) activity was measured using ALT assay kits (Sigma, #MAK052) and serum aspartate transaminase (AST) activities was measured using AST assay kits (Sigma, #MAK055) following the manufacturer's instructions. Briefly, serum samples at different dilutions, were reacted with ALT or AST reaction mixture and plated on 96-well plate, followed by reading at A450. The final calculations were done by comparing the sample readings with that of standard curve (pyruvate standards for ALT and glutamate standards for AST).

### Tumor Size Estimation in Human Brain

SJ-GBM2-luc cells at a density of 10², 10³, 10⁴, 10⁵, 10⁶ and 10⁷ cells/well in a 96-well were maintained by trypsinization and cell counting, followed by 5 min incubation with 5× cell lysis buffer (Promega, Cat no #E153A). Then, the cells were subjected to 100 μl D-Luciferin Potassium salt at a final concentration 150 μg/ml and the bioluminescence was observed with Spectral Lago X imaging system. Aura software was used to compute regions of interest (ROI) and integrate the total bioluminescence signal in each ROI, and standard curve was generated for ROI with known concentrations of cell number. The cell number for desired luminescent signal from ROI was measured and converted into relative tumor size in pediatric brain. The calculation was performed assuming 1 cm³ tumor contains 109 cells, mice whole brain size averages 0.58 cm³, and pediatric brain size averages 1,312 cm³.

### Cancer Cell Detection in Tissues by Alu Sequencing

A quantitative analysis using human-specific Alu sequence (hAlu) was performed on samples collected from the isolated brain tissues. Detection of the human derived tumors implanted into mice can be identified by the Alu sequences, specific to human cells. Genomic DNA was extracted from harvested tissues using the DNeasy Blood & Tissue kit (Qiagen). Real time PCR was done with primers specific for hAlu and mGAPDH (5 ng genomic DNA, 0.5 μM each primer, and PowerUp SYBR Green Master Mix; Life Technologies, USA). Each reaction was performed in a final volume of 10 μl at 50° C. for 2 min and 95° C. for 2 min followed by 30 cycles of 95° C. for 30 sec, 63° C. for 30 sec, and 72° C. for 30 sec. The amount of hAlu and mGAPDH DNA in the tissue was calculated by comparison with a standard curve, and the calculated amount of hAlu DNA was normalized against the relative quantity of mGAPDH. The results were verified by performing PCR using M13 modified forward and reverse primers specific to hAlu. The PCR products were visualized in 2% agarose gels and confirmed by DNA-sequencing. Actin was used as a loading control by using primers that recognize both mouse and human actin (521 nt-575 nt).

### List of Primers

| Primer | |
|---|---|
| name | Sequence |
| miR17-RT | GTCGTATCCAGTGCAGGGTCCGAGGTATTCGCACTGGATACGACCTACCT |
| | (SEQ ID NO: 10) |
| miR17-F | GTATACCAAAGTGCTTACAGT (SEQ ID NO: 11) |
| miR18a-RT | GTCGTATCCAGTGCAGGGTCCGAGGTATTCGCACTGGATACGACCTATT |
| | (SEQ ID NO: 12) |
| miR18a-F | GTATACTAAGGTGCATCTAGT (SEQ ID NO: 13) |
| miR19a-RT | GTCGTATCCAGTGCAGGGTCCGAGGTATTCGCACTGGATACGACTGTAGT |
| | (SEQ ID NO: 14) |
| miR19a-F | GTATACAGTTTTGCATAGTTG (SEQ ID NO: 15) |
| miR19b-RT | GTCGTATCCAGTGCAGGGTCCGAGGTATTCGCACTGGATACGACGCTGGA |
| | (SEQ ID NO: 16) |
| miR19b-F | GTATACAGTTTTGCAGGTTTG (SEQ ID NO: 17) |
| miR20a-RT | GTCGTATCCAGTGCAGGGTCCGAGGTCGGCAATTGCACTGGATACGACCTA |
| | CCT (SEQ ID NO: 18) |
| miR20a-F | GTAAAGTGCTTATAGTGCAG (SEQ ID NO: 19) |
| miR92a-RT | GTCGTATCCAGTGCAGGGTCCGAGGTATTCGCACTGGATACGACAGCATT |
| | (SEQ ID NO: 20) |
| miR92a-F | GTATACAGGTTGGGATCGGTT (SEQ ID NO: 21) |
| miR106-RT | GTCGTATCCAGTGCAGGGTCCGAGGTATTCGCACTGGATACGACCTACCT |
| | (SEQ ID NO: 22) |
| miR106-F | GTATACAAAAGTGCTTACAGT (SEQ ID NO: 23) |
| Anti-miR20- | GTCGTATCCAGTGCAGGGTCCGAGGTATTCGCACTGGATACGACTAAAGT |
| RT | (SEQ ID NO: 24) |
| Anti-miR20- | GTATACCTACCTGCACTATAA (SEQ ID NO: 25) |
| F | |
| U6-RT | GTCGTATCCAGTGCGTGTCGTGGAGTCGGCAATTGCACTGGATACGACAAA |
| | ATATG (SEQ ID NO: 26) |
| U6-F | TTAGCATGGCCCCTGCG (SEQ ID NO: 27) |
| Univ-R | GTGCAGGGTCCGAGGT (SEQ ID NO: 28) |
| EZH2-F | TACTTGTGGAGCCGCTGAC (SEQ ID NO: 29) |
| EZH2-R | TGCCAGAGAAGAGTGTGGTG (SEQ ID NO: 30) |
| E2F-F | CGGCGCATCTATGACATCAC (SEQ ID NO: 31) |
| E2F-R | GTCAACCCCTCAAGCCGTC (SEQ ID NO: 32) |
| ELK-1-F | CATCATCTCCTGGACCTCACG (SEQ ID NO: 33) |
| ELK-1-R | ACCTCAGGCTGGGGGGGCAGTCTT (SEQ ID NO: 34) |
| c-Jun-F | CGGACCGTTCTATGACTGC (SEQ ID NO: 35) |
| c-Jun-R | AGCGTGTTCTGGCTATGC (SEQ ID NO: 36) |
| c-Fos-F | CGGGTTTCAACGCCGACTACG (SEQ ID NO: 37) |
| c-Fos-R | GCAACGCAGACTTCTCATC (SEQ ID NO: 38) |
| c-Myc-F | CGGAACTCTTGTGCGTAAGG (SEQ ID NO: 39) |
| c-Myc-R | CTCAGCCAAGGTTGTGAGGT (SEQ ID NO: 40) |
| ERK-F | GGTTGTTCCCAAATGCTG (SEQ ID NO: 41) |
| ERK-R | CTCCTTAGGTAAGTCGTCCA (SEQ ID NO: 42) |
| TGF-F | TGGCGTTACCTTGGTAACC (SEQ ID NO: 43) |
| TGF-R | GGTGTTGAGCCCTTTCCAG (SEQ ID NO: 44) |
| p53-F | AGAGTCTATAGGCCCACCCC (SEQ ID NO: 45) |
| p53-F | GCTCGACGCTAGGATCTGAC (SEQ ID NO: 46) |
| p21-F | AAGACCATGTGGACCTGT (SEQ ID NO: 47) |
| p21-R | GGTAGAAATCTGTCATGCTG (SEQ ID NO: 48) |
| Bcl2-F | GATGTGATGCCTCTGCGAAG (SEQ ID NO: 49) |
| Bcl2-R | CATGCTGATGTCTCTGGAATCT (SEQ ID NO: 50) |
| PTEN-F | ACCATAACCCACCACAGC (SEQ ID NO: 51) |
| PTEN-R | CAGTTCGTCCCTTTCCAG (SEQ ID NO: 52) |
| hGAPDH-F | ACGGATTTGGTCGTATTGGG (SEQ ID NO: 53) |
| hGAPDH-R | CGCTCCTGGAAGATGGTGAT (SEQ ID NO: 54) |
| mGAPDH-F | AGGTCGGTGTGAACGGATTTG (SEQ ID NO: 55) |
| mGAPDH-R | GGGGTCGTTGATGGCAACA (SEQ ID NO: 56) |
| M13-hAlu-F | GTAAAACGACGGCCAACGCCTGTAATCCCAGCACTT (SEQ ID NO: 57) |
| M13-hAlu-R | GTAAAACGACGGCCA TCGCCCAGGCTGGAGTGCA (SEQ ID NO: 58) |
| M13- | GTAAAACGACGGCCAGTGTAAAGTGCTTATAGTGCAG (SEQ ID NO: 59) |
| miR20a-F | |
| M13- | GTAAAACGACGGCCAGTGTATACCTACCTGCACTATAA (SEQ ID NO: 60) |
| AmiR20a-F | |
| M13-R | AACAGCTATGACCATGGTGCAGGGTCCGAGGT (SEQ ID NO: 61) |
| Actin-F | CGTTTCATGACCAC (SEQ ID NO: 62) |
| Actin-R | GTAGCAGGGGGATC (SEQ ID NO: 63) |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A composition comprising an antisense microRNA covalently linked to a cell penetrating peptide, the cell penetrating peptide comprising p28.

2. The composition of claim 1, wherein the antisense microRNA is an antisense miR20, an antisense miR21, or an antisense miR21a.

3. The composition of claim 1, wherein the covalent linkage occurs between a maleimide-antisense microRNA and a Cysteine residue of p28 through a maleimide-thiol reaction.

4. The composition of claim 1, wherein the covalent linkage occurs between a hexynyl-antisense microRNA and an azide-p28.

5. The composition of claim 1, wherein the cell penetrating peptide comprises at least 90% sequence identity to p28.

6. A method of inhibiting tumor cell growth comprising systemic administration to the tumor cell of a composition of claim 1, comprising an antisense microRNA covalently linked to a cell penetrating peptide, the cell penetrating peptide comprising p28.

7. The method of claim 6, wherein the antisense microRNA is an antisense miR20, an antisense miR21, or an antisense miR21a.

8. The method of claim 6, wherein the covalent linkage occurs between a maleimide-antisense microRNA and a Cysteine residue of p28 through a maleimide-thiol reaction or the covalent linkage occurs between a hexynyl-antisense microRNA and an azide-p28.

9. The method of claim 6, wherein the cell penetrating peptide comprises at least 90% sequence identity to p28.

10. A method of targeting a microRNA to a tumor cell comprising administration to the tumor cell of a composition comprising an antisense microRNA covalently linked to a cell penetrating peptide, the cell penetrating peptide comprising p28.

11. The method of claim 10, wherein the antisense microRNA is an antisense miR20, an antisense miR21, or an antisense miR21a.

12. The method of claim 10, wherein the covalent linkage occurs between a maleimide-antisense microRNA and a Cysteine residue of p28 through a maleimide-thiol reaction or the covalent linkage occurs between a hexynyl-antisense microRNA and an azide-p28.

13. The method of claim 10, wherein the cell penetrating peptide comprises at least 90% sequence identity to p28.

14. The method of claim 10, wherein the cell is an in vivo cell.

15. The method of claim 10, wherein the antisense microRNA covalently linked to a cell penetrating peptide forms a complex that crosses the blood-brain-barrier.

16. A method of reducing endogenous microRNA in a tumor cell comprising systemic administration to the tumor cell of a composition of claim 1, comprising an antisense microRNA covalently linked to a cell penetrating peptide, the cell penetrating peptide comprising p28.

17. The method of claim 16, wherein the tumor cell is an in vivo cell.

18. The method of claim 16, wherein the antisense microRNA is an antisense miR20, an antisense miR21, or an antisense miR21a.

19. A method of assessing pGBM tumor status in a subject comprising:
providing a chart that indicates a correlation between miRNA concentration and tumor presence and size of tumor;
obtaining a liquid biopsy sample from the subject;
measuring miR20 concentration in the liquid biopsy sample;
comparing the concentration of miR20 in the liquid biopsy sample and the chart,
wherein the concentration of miR20 is indicative of the presence and size of tumor and results in an assessment of pGBM tumor status.

20. The method of claim 19, wherein the step of measuring miR20 comprises use of an antisense microRNA covalently linked to a cell penetrating peptide and a covalently linked to a detectable label, the cell penetrating peptide comprising p28.
